(19) United States
(12) Patent Application Publication
(10) Pub. No.: US 2026/0267951 A1
(43) Pub. Date: Sep. 10, 2026

(54) APPARATUS AND METHOD FOR PREVENTING YOUTH ACCESS AND COUNTERFEIT AEROSOL DELIVERY

(71) Applicant: SkyX IP Holdings I LLC, New York, NY (US)

(72) Inventors: Martin Steinbauer, New York, NY (US); Kylie Halperin, New York, NY (US); Xuedan Long, Shenzhen City (CN); Kristine Bunker Moore, Cambridge, MA (US); Cole Constantineau, Cambridge, MA (US); Kyle Rawding, Cambridge, MA (US); Sylvere Azoulai, Mamaroneck, NY (US)

(73) Assignee: SkyX IP Holdings I LLC, New York, NY (US)

(21) Appl. No.: 19/543,253

(22) Filed: Feb. 18, 2026

Related U.S. Application Data
(63) Continuation of application No. 18/410,193, filed on Jan. 11, 2024.

(51) Int. Cl. G06F 21/32; A61M 15/00

(52) U.S. Cl. CPC G06F 21/32; A61M 15/009; A61M 2205/8206

(57) ABSTRACT

An apparatus for preventing youth access and counterfeit aerosol delivery, comprising a cartridge, wherein the cartridge includes at least one reservoir configured to store an aerosolizable material, an aerosol delivery mechanism configured to generate aerosol using the aerosolizable material stored in the at least one reservoir, and an electrical interface having a resistor associated with a unique identifier, and a device having at least a sensor and a processing circuit configured to detect a sensed datum pertaining to a user using the at least a sensor, read the unique identifier associated with the resistor upon an electrical connection of the cartridge and the device through the electrical interface, validate the sensed datum and the unique identifier, and activate the aerosol delivery mechanism of the cartridge as a function of a positive validation of the sensed datum and the unique identifier.

### CROSS-REFERENCE TO RELATED APPLICATIONS

This application is a continuation of U.S. patent application Ser. No. 18/410,193, filed on Jan. 11, 2024, and titled “APPARATUS AND METHOD FOR PREVENTING YOUTH ACCESS AND COUNTERFEIT AEROSOL DELIVERY,” of which is incorporated by reference herein in its entirety.

### FIELD OF THE INVENTION

The present invention generally relates to the field of aerosol delivery. In particular, the present invention is directed to an apparatus and method for preventing youth access and counterfeit aerosol delivery cartridges.

### BACKGROUND

More than 1 billion people smoke globally and at least 70% of smokers want to quit. A low efficacy of FDA approved smoking cessation treatment leads to many quit attempts per person before successfully stopping to smoke. Annually 8 million people die prematurely from the use of tobacco products, and 1 billion people are projected to die early this century from tobacco use. Aerosol delivery systems allow for a safe administration of nicotine and have been proved in clinical studies to at least double the chances of smokers to quit smoking. However, cartridges for aerosol delivery systems produced by unauthorized companies may come with a host of potential issues that can compromise the user safety and experience, while also allowing access for youth to use the product. Similarly, counterfeit devices may offer unauthorized users to use genuine cartridges. As such, a solution for validating the authenticity of both the device and the cartridge is needed for aerosol delivery systems to reach their full public health potential.

### SUMMARY OF THE DISCLOSURE

In an aspect, an apparatus for preventing counterfeit aerosol delivery is described. The apparatus includes a cartridge having at least one reservoir configured to store an aerosolizable material, an aerosol delivery mechanism configured to generate aerosol using the aerosolizable material stored in the at least one reservoir, and an electrical interface having a resistor associated with a unique identifier. The apparatus further includes a device, wherein the device includes at least a sensor and a processing circuit communicatively connected to the at least a sensor, wherein the processing circuit is configured to detect a sensed datum pertaining to a user using the at least a sensor, read the unique identifier associated with the resistor upon an electrical connection of the cartridge and the device using through the electrical interface, validate the sensed datum and the unique identifier, and activate the aerosol delivery mechanism of the cartridge as a function of a positive validation of the sensed datum and the unique identifier.

In another aspect, a method for preventing counterfeit aerosol delivery is described. The method includes electrically connecting, through an electrical interface having a resistor, a cartridge, and a device, wherein the cartridge includes at least one reservoir configured to store an aerosolizable material and an aerosol delivery mechanism configured to generate aerosol using the aerosolizable material stored in the at least one reservoir, and wherein the device includes at least a sensor. The method further includes detecting, by a processing circuit, a sensed datum pertaining to a user using the at least a sensor, reading, by the processing circuit, a unique identifier associated with the resistor, validating, by the processing circuit, the sensed datum, and the unique identifier, and activating, by the processing circuit, the aerosol delivery mechanism of the cartridge as a function of a positive validation of the sensed datum and the unique identifier.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1A is an exemplary embodiment of a cartridge;

FIG. 1B is an exemplary embodiment of a bottom side view of bottom housing having an electrical interface;

FIG. 1C is an exemplary embodiment of a PCB of a cartridge;

FIG. 1D is a cross section view of an exemplary embodiment of bottom housing;

FIG. 1E is an exploded view of an exemplary embodiment of a device;

FIG. 1F is a block diagram of an exemplary embodiment of apparatus 100;

FIG. 2 is a schematic of an exemplary embodiment of a device circuitry;

FIG. 3 depicts and exemplary embodiment of an outer body of a device;

FIG. 4 depicts an example embodiment of a bottom housing of a pod;

FIG. 5 is exploded view of an exemplary embodiment of an aerosol delivery device with a cartridge integrated into device;

FIG. 6 is an exemplary embodiment of an immutable sequential listing;

FIG. 7 is a block diagram of an exemplary embodiment of a machine-learning module;

FIG. 8 is a diagram of an exemplary embodiment of a neural network;

FIG. 9 depicts an exemplary embodiment of a node of a neural network;

FIG. 10 depicts a flow diagram of an exemplary method for preventing counterfeit aerosol delivery; and

FIG. 11 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for preventing counterfeit aerosol delivery. In an embodiment, apparatus may utilize both device unique identifier and user biometric data to ensure device security and safe aerosol delivery. Exemplary embodiments illustrating aspects of the present disclosure are described below in the context of several specific examples.

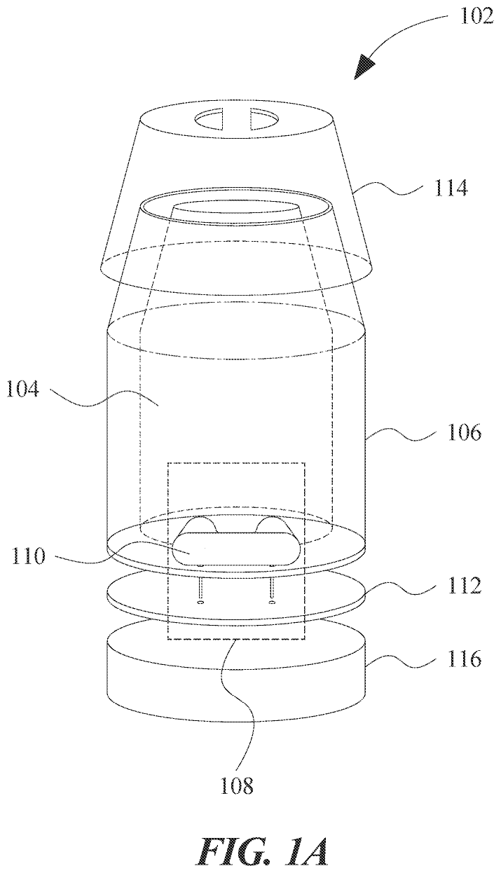

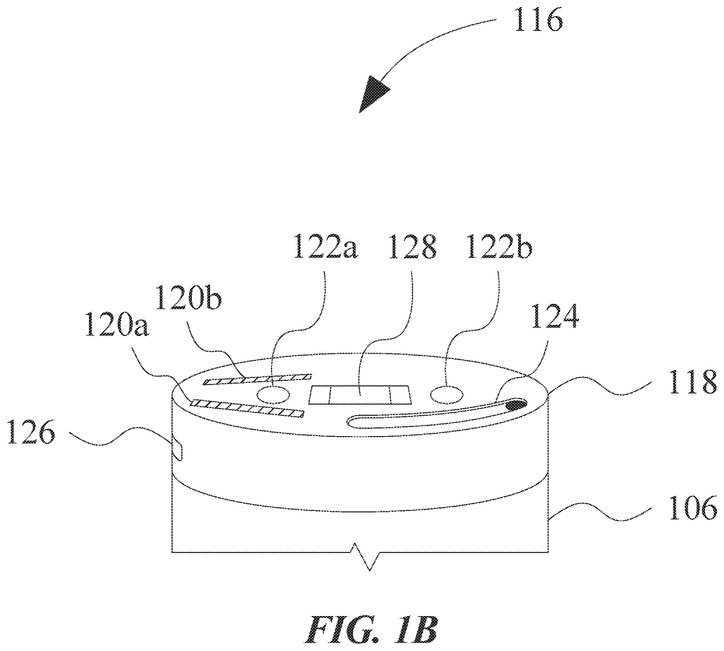

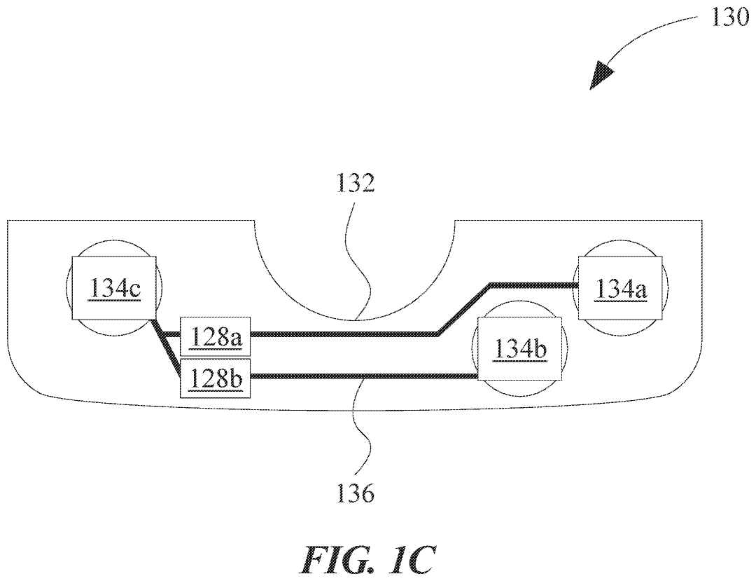

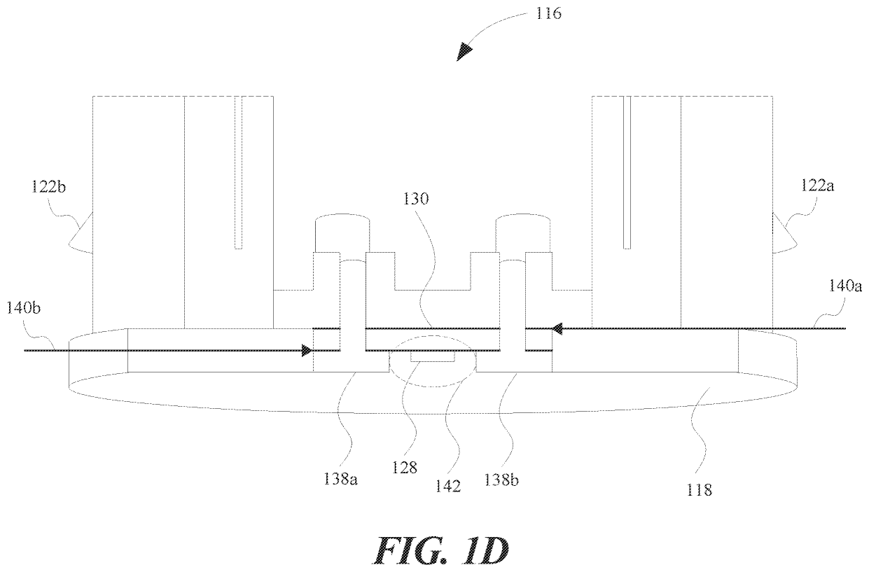

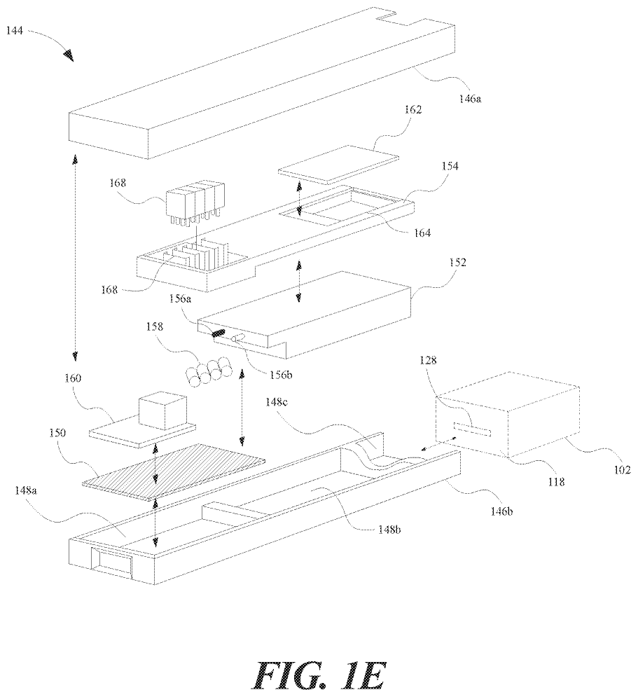

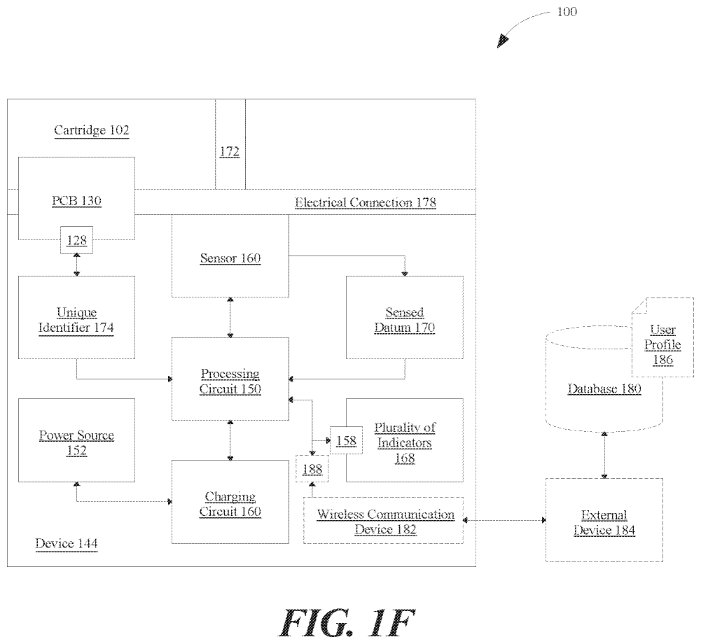

Now referring to FIGS. 1A-D, an exemplary embodiment of an apparatus 100 for preventing counterfeit aerosol delivery is illustrated. A “counterfeit aerosol delivery,” for the purpose of this disclosure, is an unauthorized or imitation system, apparatus, or method that falsely claims or appears to produce aerosolized substances, typically for inhalation, but does not meet pre-defined standards, specifications, or functionalities of an authentic or genuine aerosol delivery system. Such counterfeit systems may compromise the quality, safety, and efficacy of the aerosol produced, potentially posing risks to users. In an embodiment, counterfeit aerosol delivery may include a material-based counterfeit, wherein the material-based counterfeit aerosol delivery system may use imitation or substandard aerosolizable materials; for instance, and without limitation, instead of using a genuine nicotine solution, a counterfeit system may use a solution with impurities, without the claimed nicotine concentration, or is not officially sold. In another embodiment, counterfeit aerosol delivery may include a mechanism-based counterfeit. In a non-limiting example, instead of a genuine cartridge that vaporizes the material efficiently, the counterfeit may use a non-genuine or subpar cartridge that is incompatible (e.g., imperfect fit, inefficient aerosol generation, aerosol delivery mechanism malfunction, safety features malfunction, etc.,) with authentic devices.

Now referring to FIG. 1A, an exemplary embodiment of a cartridge 102 in an exploded view is illustrated. apparatus 100 includes a cartridge 102. As used in this disclosure, a “cartridge” is a component designed to fit into or attach to a larger device and is configured to store and deliver a specific substance or material. In an embodiment, cartridge 102 may include a compact, replaceable container that contains an aerosolizable material 104, wherein the “aerosolizable material,” for the purpose of this disclosure, is a material that is capable for aerosolization (i.e., a process of intentionally oxidatively converting and suspending particles or a composition in a moving stream of air). Cartridge 102 includes at least one reservoir 106 configured to store aerosolizable material 104. In some cases, at least one reservoir 106 may include a transparent, semi-transparent, or otherwise non-transparent main housing configured to hold at least one type of aerosolizable material. In some cases, cartridge 102 include a top housing that encloses or partially encloses the at least one reservoir 106. As used in this disclosure, a “top housing” is an upper component or section of cartridge 102 that forms a protective and structural cover over the internal components, primarily the at least one reservoir where the aerosolizable material 104 is stored. In an embodiment, top housing may be transparent or semitransparent, allowing user to visually monitor, for example, the level of aerosolizable material 104 in the at least one reservoir 106.

With continued reference to FIG. 1A, in some cases, aerosolizable material may include one or more active ingredients and/or chemicals, including without limitation pharmaceutical chemicals, recreational chemicals, flavor-bearing chemicals, and the like. Chemicals may be extracted, without limitation, from plant material, and/or a botanical, such as tobacco or other herbs or blends. Chemicals may be in pure form and/or in combination or mixture with humectants that may or may not be mixed with plant material. In a non-limiting example, aerosolizable material may include E-cigarette liquid, wherein the E-cigarette liquid is a liquid solution or mixture used in aerosol delivery device such as, without limitation, an e-cigarette.

With continued reference to FIG. 1A, in some cases, aerosolizable material may include a humectant, wherein the “humectant” may generally refer to as a substance that is used to keep things moist. Humectant may attract and retain moisture in the air by absorption, allowing the water to be used by other substances. Humectants are also commonly used in many tobaccos or botanicals and electronic vaporization products to keep products moist and as vapor-forming medium. Examples may include, without limitation, propylene glycol, sugar polyols such as glycerol, glycerin and the like thereof. Continuing the non-limiting example, E-cigarette liquid may consist a combination of propylene glycol and glycerin (95%), and flavorings, nicotine, and other additives (5%).

With continued reference to FIG. 1A, in some cases, aerosolizable material 104 held by at least one reservoir 106 may be replaceable. In a non-limiting example, at least one reservoir 106 may include a secondary container such as a liquid chamber, wherein the liquid chamber may contain a single type of aerosolizable material. Liquid chamber may be inserted into aerosolizable material reservoir; in other words, aerosolizable material 104 may not be in direct contact with at least one reservoir 106. User of apparatus 100 may switch from a first aerosolizable material to a second aerosolizable material by ejecting a first liquid chamber storing the first aerosolizable material from reservoir 106 and inserting a second liquid chamber storing the second aerosolizable material into reservoir 106. In some cases, whole cartridge 102 may be replaceable.

With continued reference to FIG. 1A, cartridge 102 includes an aerosol delivery mechanism 108. As used in this disclosure, an “aerosol delivery mechanism” is a component of apparatus 100 configured to generate aerosol using aerosolizable material. In an embodiment, aerosol delivery mechanism may be configured to convert any aerosolizable material 104 as described herein into a vapor. “Vapor,” for the purpose of this disclosure, is a substance that is in a gas phase at a temperature lower than its critical point. The vapor may be condensed to a liquid or to a solid by increasing its pressure without reducing the temperature. Vapor may include an aerosol i.e., a colloid of fine solid particles or liquid droplets in air or another gas. Examples of aerosols may include clouds, haze, and smoke, including the smoke from tobacco or botanical products.

With continued reference to FIG. 1A, in some cases, liquid or solid particles in an aerosol may have varying diameters of average mass that may range from monodisperse aerosols, producible in the laboratory, and containing particles of uniform size; to polydisperse colloidal systems, exhibiting a range of particle sizes. As the sizes of these particles become larger, they have a greater settling speed which causes them to settle out of the aerosol faster, making the appearance of the aerosol less dense and to shorten the time in which the aerosol will linger in air. Interestingly, an aerosol with smaller particles will appear thicker or denser because it has more particles. Particle number has a much bigger impact on light scattering than particle size (at least for the considered ranges of particle size), thus allowing for a vapor cloud with more smaller particles to appear denser than a cloud having fewer, but larger particle sizes.

With continued reference to FIG. 1A, in some cases, aerosol delivery mechanism 106 may include a heating element 110, which may include a resistive heater configured to thermally contact aerosolizable material 104 from reservoir 106. A Power source as described in detail below may provide electricity to heating element 110. In a non-limiting example, using heating element of aerosol delivery mechanism 120 for vaporization of aerosolizable material 104 may be used as an alternative to burning (smoking) which may avoid inhalation of many irritating and/or toxic carcinogenic byproducts which may result from pyrolytic processes of burning material such as, without limitation, tobacco or botanical products above 300 degrees C. Heating element may operate at a temperature at/or below 300 degrees C. In a non-limiting example, aerosol delivery mechanism 106 may include at least a coil and a wick surround or threaded through it. In some cases, aerosol delivery mechanism may also include a seal 112. In a non-limiting example, coil may be heat up when electric current passes through it, vaporizing aerosolizable material 104 absorbed by the wick, wherein the seal may ensure that aerosolizable material 104 does not leak out of cartridge 102 during vaporization.

With continued reference to FIG. 1A, in a non-limiting example, aerosol delivery mechanism 108 may include an atomizer and/or cartomizer configured to heat aerosolizable material 104. As used in this disclosure, an “atomizer” is a device for emitting liquid, such as aerosolizable material 104, as a fine spray such as, without limitation, a vapor. Aerosolizable material 104 may include any aerosolizable material described above in this disclosure; for instance, and without limitation, aerosolizable material may comprise glycerin and/or propylene glycol. Atomizer may be a device or system configured to generate an aerosol. An atomizer may include, without limitation, a small heating element that heats and/or vaporizes at least a portion of aerosolizable material 104 and a wicking material e.g., the wick that may draw a liquid aerosolizable material 104 in to the atomizer; a wicking material may comprise silica fibers, cotton, ceramic, hemp, stainless steel mesh, and/or rope cables. A wicking material may be designed and/or configured to draw liquid aerosolizable material into atomizer without a pump or other mechanical moving part. A resistance wire may be wrapped around a wicking material or integrated into the wicking material and then connected to a positive and negative pole of a current source such as a power source as noted above; a resistance wire may include, without limitation, a coil, and when activated may have a temperature increase as a result of the current flowing through the resistive wire to generate heat. Heat may be transferred from heating element 110 to aerosolizable material 104 through conductive, convective, and/or radiative heat transfer such that aerosolizable material vaporizes.

With continued reference to FIG. 1A, in other cases, aerosol delivery mechanism 108 may be an oven instead, which may be at least partially closed. An “oven,” for the purpose of this disclosure, is a component configured to heat confined substances, such as, without limitation, aerosolizable material 104. Oven may have a closable opening. Oven may be wrapped with heating element 110 or may be in thermal communication with a heating element by means of another mechanism. Aerosolizable material 104 may be placed directly in an oven or in a liquid chamber fitted in the oven. A heating element 110 in thermal communication with the oven may heat aerosolizable material mass in order to create a gas phase vapor, including without limitation through conductive, convective, and/or radiative heat transfer. Vapor may be released to a vaporization chamber where gas phase vapor may condense, forming an aerosol cloud having typical liquid vapor particles with particles having a diameter of average mass of approximately 1 micron or greater. In some cases, the diameter of average mass may be approximately 0.1-3 micron.

With continued reference to FIG. 1A, air may be drawn into cartridge 102 and/or aerosol delivery mechanism 108 to carry the vaporized aerosol away from heating element 110, where it then cools and condenses to form liquid particles suspended in air. In some cases, air may flow to printed circuit board (PCB) of cartridge 102 to assist in thermal regulation as described in detail below. In some cases, directing airflow over the PCB may aid in dissipating heat generated due to the operation of PCB. In a non-limiting example, a vapor tube (i.e., a conduit designed to channel the vaporized aerosol from the heating element to a mouthpiece) and an airflow tube (i.e., a separate conduit designed to guide the incoming air towards the heating element and PCB) may be molded at each side of cartridge 102. In some cases, these tubes may be symmetrical, ensuring an even distribution of airflow, or they may have varied dimensions to prioritize either the vapor flow or the cooling airflow.

With continued reference to FIG. 1A, in some cases, cartridge 102 may include a mouthpiece 114. As used in this disclosure, a “mouthpiece” is a component configured to facilitate inhalation of the aerosol produced by aerosol delivery mechanism 108 within cartridge 102. In some cases, air may be drawn out of mouthpiece 114 by a user. In a non-limiting example, mouthpiece may include one or more air holes, wherein at least an air hole is configured as a passage that allows air to pass through apparatus 100. In an embodiments, fresh air may be allowed to enter apparatus 100 and aerosol delivery mechanism 108 when heating element 110 is on. In some cases, mouthpiece 114 may include a one-way valve to prevent any backflow of air or moisture into cartridge 102. In some cases, mouthpiece 114 may include an attachment. In some cases, mouthpiece 114 may be ergonomically designed to fit the contours of the user's lips. In some cases, mouthpiece 114 may be constructed from materials that are safe for oral contact, e.g., food-grade plastics as described herein. In a non-limiting example, mouthpiece 114 may be detachable, allowing user to clean or replace it as needed. Mouthpiece 114 may include any mouthpiece as described in U.S. patent application Ser. No. 18/370,272 (Attorney docket number 1445-005USU1), filed on Sep. 19, 2023, and entitled “RIDGED MOUTHPIECE FOR AEROSOL DELIVERY DEVICES,” which its entirety is incorporated herein by reference.

With continued reference to FIG. 1A in some cases, cartridge 102 may include a bottom housing 116. As used in this disclosure, a “bottom housing” is a foundation structure or base of cartridge 102. In an embodiment, bottom housing 116 may be designed to enclose and protect internal components such as, without limitation, at least one reservoir 106, aerosol delivery mechanism 108, and the like. In some cases, bottom housing 116 and mouthpiece 114 may be positioned and attached at opposite ends of cartridge 102. In a non-limiting example, bottom housing 116 and mouthpiece 114 may be situated at a proximal end and a distal end, respectively. In some cases, aerosol delivery mechanism 108 may be centrally located within bottom housing 116. In some cases, aerosol delivery mechanism may be situated along (at least a portion of) perimeter of bottom housing 116. In a non-limiting example, in case of a multi-flavor cartridge i.e., cartridge with multiple separated reservoirs, aerosolizable materials may be selectively heated by activating aerosol delivery mechanism at specific sections. In some cases, aerosol delivery mechanism 108 may be recessed or embedded into bottom housing 116. In some cases, bottom housing 116 may include an elevated platform for placing aerosol delivery mechanism 108. This may prevent potential clogging of condensed or residual liquid from clogging at heating element 110 or heating element 110 malfunction. In other cases, aerosol delivery mechanism 108 may be integrated with one or more airflow channels. Bottom housing is described in further detail below with reference to FIG. 1B and FIG. 1D.

With continued reference to FIG. 1A, in some cases, vaporization of aerosolizable material may occur at lower temperatures in aerosol delivery mechanism 108 compared to temperatures required to generate an inhalable vapor in an actual cigarette (in which a smokable material is burned to generate an inhalable vapor). The lower temperature of aerosol delivery mechanism 108 may result in less decomposition and/or reaction of aerosolizable material 104, and therefore produce an aerosol with many fewer chemical components compared to actual cigarette. In some cases, aerosol delivery mechanism 108 may generate aerosol with fewer chemical components that may be harmful to human health compared to actual cigarette.

With continued reference to FIG. 1A, exemplary embodiments of cartridge 102 may include, without limitation, E-liquid cartridge, dry herb cartridge, oil or concentrate cartridge, dual or multi-reservoir cartridge, disposable cartridge, refillable cartridge, or the like. As a person skilled in the art, upon reviewing the entirety of this disclosure, will be aware of various types of a cartridge 102 may be compatible and used by apparatus 100 as described herein.

With continued reference to FIG. 1A, in some embodiments, components of cartridge 102 may be constructed from an injectable mold. In some cases, plastic material such as, without limitation, BIOGRADE B-M (i.e., blend of thermoplastic starch (TPS), aliphatic polyesters (AP) and natural plasticizers (glycerol and sorbitol)) may be injected into the injectable mold under high pressure, filling the space and taking on the shape of injectable mold. Other exemplary plastic materials may include, without limitation, BIOPAR FG MO (i.e., bio-plastic resin consisting mainly of thermoplastic potato starch, biodegradable synthetic copolyesters and additives), BIOPLAST (i.e., new kind of plasticizer cherfreien thermoplastic material), ENSO RENEW RTP (i.e., renewable, biodegradable, compostable and economic thermoplastic), and/or the like. As an example, components such as 106, 108, 112, 114, 116 and the like may be made with biodegradable or compostable plastics, and the heating element could be made of cotton. The majority of the cartridge may be biodegradable. As the cartridge is replaceable and has a short life span for a user, there is either a need to recycle the cartridge, or more preferably to biodegrade under certain conditions such as composting in soil over a duration of time. As different components have different functions and require specific properties, such as heat or chemicals resistance for the oven, the correct plastics need to be chosen for their moldability and chemical/material properties. Furthermore, for plastics in contact with the aerosolizable material or the user, extractables and leachable testing is required to determine the most optimal composition. In one embodiment, all materials apart from electrical contacts, heating wire, and resistor/PCB may be biodegradable yielding a biodegradability rate above 80% for cartridge 102. In one embodiment, cartridge is made of at least 50% biodegradable/compostable plastics by volume.

Now referring to FIG. 1B, an exemplary embodiment of a bottom side view of bottom housing 116 having an electrical interface 118 is illustrated. As used in this disclosure, an “electrical interface” is a system or component that facilitates the transfer of electrical signals or power between two or more devices, components, or systems. In an embodiment, electrical interface 118 may serve as a point of connection or communication that ensures compatibility and proper functioning between cartridge 102 and the device as described below. In some cases, electrical interface may be configured to transmit signal (data), power, or both. In some cases, electrical interface may be the bottom surface of bottom housing 116 as described above.

With continued reference to FIG. 1B, as used in this disclosure, a “signal” is any intelligible representation of data, for example from one device to another. A signal may include an optical signal, a hydraulic signal, a pneumatic signal, a mechanical signal, an electric signal, a digital signal, an analog signal and the like. In some cases, a signal may be used to communicate with a computing device, for example by way of one or more ports. In some cases, a signal may be transmitted and/or received by a computing device for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

With continued reference to FIG. 1B, in some cases, electrical interface 118 may include a plurality of contacts 120a-b. As used in this disclosures, “contacts” are conductive elements or points designed to establish an electrical connection with corresponding elements another device or component. In a non-limiting example, plurality of contacts may be made of material with high electrical conductivity e.g., copper. Bottom side of bottom housing 116 may include a plurality of contact slots, each designed to securely accommodate and align with one of plurality of contacts 120a-b, ensuring that plurality of contacts 120a-b are correctly positioned for desired electrical connection when cartridge 102 is attached to device or main body of apparatus 100. In some cases, contact slots may provide a snug fit for minimizing the risk of disconnection or misalignment during use. In some cases, plurality of contacts 120a-b may be designed with specific shapes, sizes, or configurations as a form of mechanical keying. In an embodiment, plurality of contacts 120a-b may include long contacts. Other exemplary embodiments of contacts 120a-b that are dedicated to power transfer and/or data communication may include dome switches, spring contacts (e.g., leaf spring contacts), and/or the like.

With continued reference to FIG. 1B, in some cases, plurality of contacts 120a-b may be arranged in a linear arrangement i.e., a straight line, equidistant from each other. In some cases, plurality of contacts may be in a circular arrangement i.e., around perimeter of the bottom housing's 116 base. In a non-limiting example, cartridge 102 may be inserted in any orientation, allowing for a 360-degree connection capability. Additionally, or alternatively, one or more orientation markings or notches may be presented on electrical interface 118, near plurality of contacts 120a-b, to aid users in correctly inserting cartridge 102. In some cases, orientation markings may be aligned with indicators on device as described below. In some cases, to prevent accidental short-circuits or damage, plurality of contacts 120a-b may be slightly recessed into bottom housing 116.

With continued reference to FIG. 1B, in some cases, electrical interface 118 may include one or more mechanical interfaces 122a-b configured to ensure a secure and stable connection between cartridge and device. As used in this disclosure, a “mechanical interface” is a component designed to facilitate such attachment, alignment, or connection of two parts. In some cases, mechanical interfaces 122a-b may complement the electrical connections as described herein. In an embodiment, mechanical interfaces 122a-b may include a male interface and a female interface, wherein the male interface is designed to fit snugly into the female interface. In some cases, male interface may be situated on bottom housing 116, while female interface may be located on a proximal end of device, or vice versa. In a non-limiting example, mechanical interfaces 122a-b may include at least two steel discs (i.e., magnetic discs) placed strategically on the bottom housing. These discs can be magnetically attracted to corresponding magnets or metal parts on the device. In some cases, at least two steel discs may be symmetrically placed on bottom housing's 116 base at both sides (left and right) to ensure a balanced distribution of magnetic force across the interface. Other exemplary embodiments of mechanical interface may include, without limitation, press fit, clip-in mechanism, threaded connection, and any combination thereof.

With continued reference to FIG. 1B, in some cases, bottom housing's 116 base may include one or more etched channels 124, wherein “etched channels,” for the purpose of this disclosure, are grooves or pathways that are carved or formed into material of bottom housing 116, or otherwise integrated directly into the original mold used for creating bottom housing 116. In some cases, at least one etched channel may be configured to direct airflow to PCB (for cooling PCB during operation) or for airflow activation of device by inducing a pressure change thereby activating the pressure sensor. In some cases, one or more etched channels may be configured to provide airflow to the heating element 110 to carry produced aerosol from aerosolizable material 104 to the user. In some embodiments, one end of at least one etched channel may be connected to an airflow tube, which allows air to be drawn into the cartridge. The other end of the channel may be connected to a vapor tube, which guides the vaporized aerosol towards mouthpiece 114 for inhalation.

With continued reference to FIG. 1B, additionally, or alternatively, bottom housing 116 may include a side inlet 126 (i.e., openings or vents designed to allow air to enter the cartridge from the sides of cartridge 102). In some cases, side inlet 126 may be configured to ensure a proper airflow within cartridge 102, especially when the main airflow path is obstructed or restricted. In an embodiment, side inlet 126 may include at least an aperture, such as a hole or opening that controls the amount and direction of the airflow. In some cases, the size, shape, and positioning of the aperture may be optimized to achieve desired airflow characteristics, ensuring consistent vapor generation, and preventing potential issues like “spit-back” or flooding of heating coil. In a non-limiting example, bottom housing 116 may include a plurality of side inlets. Plurality of side inlets might be positioned at opposite each of bottom housing 116 to ensure even distribution of air within cartridge 102. In some cases, side inlet 126 may also include a mesh or a filter to prevent debris or external contaminants from entering the cartridge 102. Additionally, or alternatively, POREX porous plastic may be used as permeable venting material for button housing 116 as described herein.

With continued reference to FIG. 1B, in some cases, etched channel 124 may be directly connected to side inlet 126. In a non-limiting example, when the user takes a draw from mouthpiece 114, air enters the cartridge through one or more side inlets and is then channeled through the etched channel 124. In some cases, air may be directed towards specific components within cartridge 102, such as the PCB or heating element 110. In some cases, a wider etched channel 124 may result in a looser draw, while a narrower one may offer a tighter, more restricted draw. In a non-limiting example, etched channel 124 may be designed with a slight curve or bend (around the perimeter of bottom housing 116). In other cases, multiple smaller etched channels may branch out from a main channel, distributing the air evenly throughout cartridge 102.

With continued reference to FIG. 1B, electrical interface 118 includes a resistor 128. As used in this disclosure, a “resistor” is a passive two-terminal electrical component that implements electrical resistance as a circuit element. In some cases, resistor 128 may be configured to limit or regulate the flow of electrical current in a circuit. In some cases, plurality of resistors may be built into cartridge 102, each with a specific resistance value (i.e., unique identifier as described below). In some cases, resistor 128 may be measured by apparatus 100 as described in detail below. In some cases, resistor 128 may be configured as a safety feature, ensuring cartridge 102 operates within described electrical parameters. In a non-limiting example, resistor 128 may include a “surface mount device (SMD) resistor”. SMD resistor is a type of resistor that is designed to be mounted on the surface of a printed circuit board (PCB) rather than being inserted through holes, allowing for a more compact and efficient circuit layout. In some cases, SMD resistor may be placed at the center of electrical interface 118. In a non-limiting example, resistor 128 may include a (YAGEO) YNSC086 SMD resistor. In some cases, cartridges without resistors or resistors with incorrect resistances, may be prevented by apparatus 100 from being used. Alternatively, electrical interface 118 may include a unique pin layout for device authentication. Other mechanisms for ensuring authenticity and providing limited capabilities for data collection and fraud prevention may include passive RFID tags, which can be read by apparatus 100, and memory transfer mechanisms where data may be exchanged between cartridge 102 and apparatus 100 upon a successful connection as described in detail below. In a non-limiting example, cartridge 102 may be equipped with a near-field communication (NFC) PCBA (as described in detail below with reference to FIG. 1F), which is installed using a protective overmold, glue, and/or trapping means behind a Snap-on mouthpiece. In some cases, NFC PCBA may be positioned in such a way that it is trapped behind mouthpiece within a recess or cavity that is tight enough to prevent any undesired movement of the NFC PCBA.

Now referring to FIG. 1C, an exemplary embodiment of a PCB 130 of cartridge 102 is illustrated. In some cases, PCB 130 may include a resistance reading circuit. In some cases, resistance reading circuit may be configured to measure resistance of attached components e.g., a plurality of resistors 128a-b (e.g., SMD resistors as described above). In a non-limiting example, each resistor may include a 0201 SMD package. In some cases, resistance of other components such as heating element 110 may be measured as well. In some cases, PCB 130 may include a microcontroller, an analog-to-digital converter, and other passive components such as one or more capacitors to facilitate resistance measurements. In a non-limiting example, resistance reading circuit may be connected to processing circuit as described below. In some cases, PCB 130 may be designed in a (half) oval or rectangular shape to fit within cartridge 102 e.g., bottom casing 116. In some cases, one or more edges of PCB 130 may be slightly rounded. In some cases, PCB 130 may include a circular cut-out 132 or recess. In a non-limiting example, cut-out 132 may be designed to accommodate vapor tube transporting aerosolized material and/or an airflow tube for airflow activation of the device.

With continued reference to FIG. 1C, in some cases, PCB 130 may include multiple connection points such as 134a-c. For example, and without limitation, multiple connection points may include a first connection point e.g., a voltage input (Vin) 134a and a second connection point e.g., a ground 134b. In some cases, Vin 134a may be a primary power input of PCB 130. In a non-limiting example, Vin 134a may be connected to a power source. In some cases, Vin 134a may be designed to handle voltage requirements of cartridge 102, ensuring that heating element 110 may receive necessary power for vaporization. Ground 134b may be configured as a common or return path for completing the resistance reading circuit. In some cases, ground 134b may provide a reference point for all voltage measurements, allowing for a safe operation of cartridge 102. In some cases, ground 134b may be connected to cartridge's 102 casing e.g., bottom housing 116 or a dedicated ground plane on PCB 130. In a non-limiting example, resistance reading circuit may employ a Wheatstone bridge configuration to measure resistance value associated with each resistor. In other cases, Vin 134a may be equipped with a voltage regulator. In other cases, ground 134b may be designed with multiple layers to provide an efficient heat dissipation and reduce electronic noise.

Still referring to FIG. 1C, in some cases, Vin 134a may be directly connected to first resistor 128a, wherein first resistor 128a may be configured as a part of the resistance reading circuit or potentially as a current limiting resistor as described above. For instance, and without limitation, first resistor 128a may modulate voltage supplied to PCB 130 through Vin 134a. Similarly, ground 134b may be connected to second resistor 128b, wherein second resistor 128b may act as, in some cases, a pull-down resistor. In some cases, second resistor 128b may be configured to ensure that ground potential is maintained at a consistent level. In some cases, connection between Vin 134a and the first resistor 128a, as well as the connection between ground 134b and the second resistor 128b, may be facilitated by a conductive path 136. This path, in some cases, may include a wire, a PCB trace, or any other (low-resistance) conductive medium. Both Vin 134a and ground 134b may converge at a third connection point 134c, e.g., a voltage output (Vout) 134c. In some cases, Vout 134c may be configured to provide modulated voltage to other components of cartridge 102 or device as described below. In a non-limiting example, Vout 134c may be connected to heating element 110, at least a sensor, or any other component requiring a power supply as described herein.

Now referring to FIG. 1D, a cross section view of an exemplary embodiment of bottom housing 116 is illustrated. PCB 130 may be mounted inside bottom housing 116. In a non-limiting example, a plurality of PCB brackets 138a-b may be placed within bottom housing 116 to secure PCB 130, ensure the attached PCB 130 is not subjected to unwanted movement or shifts during operation of cartridge 102. In some cases, design of PCB 130 may be bifurcated into two distinct sides, e.g., A side 140a and the B side 140b, wherein the A side 140a of PCB 130 may primarily house a majority of the circuit components such as, without limitation, microcontrollers, capacitors, diodes, and the like, and wherein the B side 140b, contrary to A side 140a, may be designed to accommodate SMD resistors 128a-b. In a non-limiting example, SMD resistors 128a-b may be placed on B side 140b to align with a SMD slot 142 present on the bottom surface of bottom housing 116. A “SMD slot,” for the purpose of this disclosure, is an aperture or slit on bottom housing 116 for one or more SMD resistors 128a-b to be inserted. In a non-limiting example, bottom housing 116 may include a rectangular hole allowing SMD resistors 128a-b from B side 140b of PCB 130 to fit in seamlessly. Additionally, or alternatively, mechanical interfaces 122a-b e.g., small clips or notches that snap into corresponding slots or grooves on device, may be situated on the side of bottom housing 116.

Now referring to FIG. 1E, an exemplary embodiment of a device 144 in an exploded view of is illustrated. Apparatus 100 includes a device 144, wherein the “device,” for the purpose of this disclosure, is a component or module designed to interface with cartridge 102 as described above, facilitating the aerosol delivery or execution of specific function related to aerosol delivery as described below. In a non-limiting example, (distinguished from cartridge 102) device 144 may include a main body or chassis of the described apparatus 100. Device 144 may be configured to house a plurality of devices as described in detail below. In some cases, when cartridge 102, containing aerosolizable material 104, is attached, or inserted into device, combined system may become operational. Such attachment/connection may be enabled by one or more mechanical interfaces 122a-b as described above. In a non-limiting example, device may include at least one designated slot or mechanical interface where cartridge 102 is attached.

With continued reference to FIG. 1E, device 144 may include a housing 146. In some cases, housing 146 may be composed of two halves e.g., housing top 146a (i.e., an upper haft of device 144) and housing bottom 146b (i.e., a lower haft of device 144). In some cases, housing 146a-b may be molded from a durable material such as, without limitation, plastic materials. In some cases, plastic materials may include, without limitation, Polyethylene (PE, including Low-Density Polyethylene [LDPE], High-Density Polyethylene [HDPE], and Linear Low-Density Polyethylene [LLDPE]), Polypropylene (PP), Polyvinyl Chloride (PVC, including, Rigid PVC [uPVC] and Flexible PVC), Polystyrene (PS, including General Purpose Polystyrene [GPPS] and High Impact Polystyrene [HIPS]), Polyethylene Terephthalate (PET), Polybutylene Terephthalate (PBT), Polycarbonate (PC), Polyurethane (PU), Polyacrylonitrile (PAN), Polyvinylidene Fluoride (PVDF), Polyvinyl Alcohol (PVA), Polytetrafluoroethylene (PTFE), Polymethyl Methacrylate/Acrylic/Plexiglas (PMMA), Polyoxymethylene/Delrin (POM), Polyether Ether Ketone (PEEK), Polyphenylene Sulfide (PPS), Polyphenylene Oxide (PPO), Polysulfone (PSU), Polyimide (PI), Polyamide/Nylon (PA), Polyethylene Naphthalate (PEN), Polybutadiene (PBD), Polyisoprene (PIR), Polyvinyl Acetate (PVAc), Polyvinyl Butyral (PVB), Polychlorotrifluoroethylene (PCTFE), Polyvinylpyrrolidone (PVP), Ethylene-Vinyl Acetate (EVA), Ethylene Propylene Diene Monomer (EPDM), Thermoplastic Elastomers (TPE), Thermoplastic Polyurethane (TPU), Thermoplastic Olefin (TPO), Liquid Crystal Polymers (LCP), Polyaryletherketone (PAEK), Polyetherimide (PEI), among others.

With continued reference to FIG. 1E, in some cases, inner side of housing top 146a may include one or more grooves or clips designed to seamlessly fit and lock with housing bottom 146b, or vice versa. In some cases, two halves may be sealed to prevent disassembly, tampering, entry of air, entry of water, or entry of contaminates. In a non-limiting example, a rubber or silicone gasket may be placed between two halves, ensuring an airtight seal when device 144 is fully assembled. In some cases, device 144 may include one or more compartments 148a-c. In a non-limiting example, device 144 may include a first compartment 148a configured to contain a processing circuit 150, a second compartment 148b configured to house a power source 152, and a third compartment 148c configured to securely hold cartridge 102. In some cases, a compartment tray 154 may be integrated within device 144 to facilitate the organization and separation of plurality of compartments 148a-c, ensuring that each compartment is isolated from the others, preventing any potential interference or cross-contamination. In some cases, compartment tray 154 may include a slide-in mechanism to allow for easy insertion and removal.

With continued reference to FIG. 1E, in one or more embodiments, processing circuit 150 may include a circuitry; for instance, and without limitation, processing circuit 150 may include an application-specific integrated circuit (ASIC). ASIC may be communicatively connected to a memory, such as memory. Memory may include rea-only memory (ROM) and/or rewritable ROM, FPGA, or other combinational and/or sequential synchronous or non-synchronous digital circuitry to store parameters described further in this disclosure. In one or more embodiments, memory may include one or more memory devices to store data and information, such as any data as described herein. The one or more memory devices may include various types of memory including, but not limited to, volatile and non-volatile memory devices, such as, for example, ROM (Read-Only Memory), EEPROM (Electrically-Erasable Read-Only Memory), RAM (Random Access Memory), flash memory, and the like. In one or more embodiments, processing circuit is adapted to execute software stored in memory to perform various methods, processes, and modes of operations in manner as described in this disclosure.

With continued reference to FIG. 1E, in a non-limiting example, processing circuit 150 may include one or more computing devices or processors. Computing device may include a processor communicatively connected to a memory. Exemplary embodiments of computing device may include, any computing device as described in this disclosure, including without limitation a microcontroller, microprocessor, digital signal processor (DSP) and/or system on a chip (SoC) as described in this disclosure. Computing device may include, be included in, and/or communicate with a remote device such as a mobile telephone, smartphone, or any other user devices. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device.

With continued reference to FIG. 1E, in some cases, network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication.

With continued reference to FIG. 1E, in general, any network topology may be used. Information (e.g., data, software etc.) may be communicated to and/or from a computer and/or a computing device. Processing circuit 150 may include a server having, but is not limited to, for example, a computing device or cluster of computing devices in a first location and a second computing device or cluster of computing devices in a second location. Server may include one or more computing devices dedicated to data storage, security, distribution of traffic for load balancing, and the like. Processing circuit 150 may distribute one or more computing tasks as described below across a plurality of computing devices of different system components such as external devices as described above, which may operate in parallel, in series, redundantly, or in any other manner used for distribution of tasks or memory between computing devices. Computing device of processing circuit 150 may be implemented, as a non-limiting example, using a “shared nothing” architecture.

With continued reference to FIG. 1E, processing circuit 150 may be designed and/or configured to perform any method, method step, or sequence of method steps in any embodiment described in this disclosure, in any order and with any degree of repetition. For instance, computing device of processing circuit 150 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks.

With continued reference to FIG. 1E, processing circuit 150 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1E, in an embodiment, methods, devices, and/or systems described herein may perform or implement one or more aspects of a cryptographic system. In one embodiment, a cryptographic system is a system that converts data from a first form, known as “plaintext,” which is intelligible when viewed in its intended format, into a second form, known as “ciphertext,” which is not intelligible when viewed in the same way. Ciphertext may be unintelligible in any format unless first converted back to plaintext. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that are “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

With continued reference to FIG. 1E, in embodiments of cryptographic systems that are “asymmetric,” either encryption or decryption key cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. An example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. Another example is elliptic curve cryptography, which relies on the fact that given two points P and Q on an elliptic curve over a finite field, and a definition for addition where A+B=−R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q.

With continued reference to FIG. 1E, in some embodiments, systems, devices, and/or methods described herein produce cryptographic hashes, also referred to by the equivalent shorthand term “hashes.” A cryptographic hash, as used herein, is a mathematical representation of a lot of data, such as files or blocks in a block chain as described in further detail below; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is a one-way function, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

With continued reference to FIG. 1E, in an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running the lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, SHA256, a NIST standard; further current and past hashing algorithms include Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-314,” and the like), Message Authentication Code (“MAC”)-family hash functions such as PMAC, OMAC, VMAC, HMAC, and UMAC, Poly 1305-AES, Elliptic Curve Only Hash (“ECOH”) and similar hash functions, Fast-Syndrome-based (FSB) hash functions, GOST hash functions, the Grostl hash function, the HAS-162 hash function, the JH hash function, the RadioGatim hash function, the Skein hash function, the Streebog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” may to detect collision may be on the order of O(2n¹²) for n output bits; thus, it may take on the order of 2²⁵⁶ operations to locate a collision in a 314 bit output “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

With continued reference to FIG. 1E, a “secure proof,” as used in this disclosure, is a protocol whereby an output is generated that demonstrates possession of a secret, such as device-specific secret, without demonstrating the entirety of the device-specific secret; in other words, a secure proof by itself, is insufficient to reconstruct the entire device-specific secret, enabling the production of at least another secure proof using at least a device-specific secret. A secure proof may be referred to as a “proof of possession” or “proof of knowledge” of a secret. Where at least a device-specific secret is a plurality of secrets, such as a plurality of challenge-response pairs, a secure proof may include an output that reveals the entirety of one of the plurality of secrets, but not all of the plurality of secrets; for instance, secure proof may be a response contained in one challenge-response pair. In an embodiment, proof may not be secure; in other words, proof may include a one-time revelation of at least a device-specific secret, for instance as used in a single challenge-response exchange.

With continued reference to FIG. 1E, secure proof may include a zero-knowledge proof, which may provide an output demonstrating possession of a secret while revealing none of the secret to a recipient of the output; zero-knowledge proof may be information-theoretically secure, meaning that an entity with infinite computing power would be unable to determine secret from output. Alternatively, zero-knowledge proof may be computationally secure, meaning that determination of secret from output is computationally infeasible, for instance to the same extent that determination of a private key from a public key in a public key cryptographic system is computationally infeasible. Zero-knowledge proof algorithms may generally include a set of two algorithms, a prover algorithm, or “P,” which is used to prove computational integrity and/or possession of a secret, and a verifier algorithm, or “V” whereby a party may check the validity of P. Zero-knowledge proof may include an interactive zero-knowledge proof, wherein a party verifying the proof must directly interact with the proving party; for instance, the verifying and proving parties may be required to be online, or connected to the same network as each other, at the same time. Interactive zero-knowledge proof may include a “proof of knowledge” proof, such as a Schnorr algorithm for proof on knowledge of a discrete logarithm. In a Schnorr algorithm, a prover commits to a randomness r, generates a message based on r, and generates a message adding r to a challenge c multiplied by a discrete logarithm that the prover is able to calculate; verification is performed by the verifier who produced c by exponentiation, thus checking the validity of the discrete logarithm. Interactive zero-knowledge proofs may alternatively or additionally include sigma protocols. Persons skilled in the art, upon reviewing the entirety of this disclosure, will be aware of various alternative interactive zero-knowledge proofs that may be implemented consistently with this disclosure.

With continued reference to FIG. 1E, alternatively, zero-knowledge proof may include a non-interactive zero-knowledge, proof, or a proof wherein neither party to the proof interacts with the other party to the proof; for instance, each of a party receiving the proof and a party providing the proof may receive a reference datum which the party providing the proof may modify or otherwise use to perform the proof. As a non-limiting example, zero-knowledge proof may include a succinct non-interactive arguments of knowledge (ZK-SNARKS) proof, wherein a “trusted setup” process creates proof and verification keys using secret (and subsequently discarded) information encoded using a public key cryptographic system, a prover runs a proving algorithm using the proving key and secret information available to the prover, and a verifier checks the proof using the verification key; public key cryptographic system may include RSA, elliptic curve cryptography, ElGamal, or any other suitable public key cryptographic system. Generation of trusted setup may be performed using a secure multiparty computation so that no one party has control of the totality of the secret information used in the trusted setup; as a result, if any one party generating the trusted setup is trustworthy, the secret information may be unrecoverable by malicious parties. As another non-limiting example, non-interactive zero-knowledge proof may include a Succinct Transparent Arguments of Knowledge (ZK-STARKS) zero-knowledge proof. In an embodiment, a ZK-STARKS proof includes a Merkle root of a Merkle tree representing evaluation of a secret computation at some number of points, which may be 1 billion points, plus Merkle branches representing evaluations at a set of randomly selected points of the number of points; verification may include determining that Merkle branches provided match the Merkle root, and that point verifications at those branches represent valid values, where validity is shown by demonstrating that all values belong to the same polynomial created by transforming the secret computation. In an embodiment, ZK-STARKS does not require a trusted setup.

With continued reference to FIG. 1E, zero-knowledge proof may include any other suitable zero-knowledge proof. Zero-knowledge proof may include, without limitation, bulletproofs. Zero-knowledge proof may include a homomorphic public-key cryptography (hPKC)-based proof. Zero-knowledge proof may include a discrete logarithmic problem (DLP) proof. Zero-knowledge proof may include a secure multi-party computation (1VIPC) proof. Zero-knowledge proof may include, without limitation, an incrementally verifiable computation (IVC). Zero-knowledge proof may include an interactive oracle proof (IOP). Zero-knowledge proof may include a proof based on the probabilistically checkable proof (PCP) theorem, including a linear PCP (LPCP) proof. Persons skilled in the art, upon reviewing the entirety of this disclosure, will be aware of various forms of zero-knowledge proofs that may be used, singly or in combination, consistently with this disclosure.

With continued reference to FIG. 1E, in an embodiment, secure proof is implemented using a challenge-response protocol. In an embodiment, this may function as a one-time pad implementation; for instance, a manufacturer or other trusted party may record a series of outputs (“responses”) produced by a device possessing secret information, given a series of corresponding inputs (“challenges”), and store them securely. In an embodiment, a challenge-response protocol may be combined with key generation. A single key may be used in one or more digital signatures as described in further detail below, such as signatures used to receive and/or transfer possession of crypto-currency assets; the key may be discarded for future use after a set period of time. In an embodiment, varied inputs include variations in local physical parameters, such as fluctuations in local electromagnetic fields, radiation, temperature, and the like, such that an almost limitless variety of private keys may be so generated. Secure proof may include encryption of a challenge to produce the response, indicating possession of a secret key. Encryption may be performed using a private key of a public key cryptographic system or using a private key of a symmetric cryptographic system; for instance, trusted party may verify response by decrypting an encryption of challenge or of another datum using either a symmetric or public-key cryptographic system, verifying that a stored key matches the key used for encryption as a function of at least a device-specific secret. Keys may be generated by random variation in selection of prime numbers, for instance for the purposes of a cryptographic system such as RSA that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem.

With continued reference to FIG. 1E, a “digital signature,” as used herein, includes a secure proof of possession of a secret by a signing device, as performed on provided element of data, known as a “message.” A message may include an encrypted mathematical representation of a file or other set of data using the private key of a public key cryptographic system. Secure proof may include any form of secure proof as described above, including without limitation encryption using a private key of a public key cryptographic system as described above. Signature may be verified using a verification datum suitable for verification of a secure proof; for instance, where secure proof is enacted by encrypting message using a private key of a public key cryptographic system, verification may include decrypting the encrypted message using the corresponding public key and comparing the decrypted representation to a purported match that was not encrypted; if the signature protocol is well-designed and implemented correctly, this means the ability to create the digital signature is equivalent to possession of the private decryption key and/or device-specific secret. Likewise, if a message making up a mathematical representation of file is well-designed and implemented correctly, any alteration of the file may result in a mismatch with the digital signature; the mathematical representation may be produced using an alteration-sensitive, reliably reproducible algorithm, such as a hashing algorithm as described above. A mathematical representation to which the signature may be compared may be included with signature, for verification purposes; in other embodiments, the algorithm used to produce the mathematical representation may be publicly available, permitting the easy reproduction of the mathematical representation corresponding to any file.

With continued reference to FIG. 1E, in some embodiments, digital signatures may be combined with or incorporated in digital certificates. In one embodiment, a digital certificate is a file that conveys information and links the conveyed information to a “certificate authority” that is the issuer of a public key in a public key cryptographic system. Certificate authority in some embodiments contains data conveying the certificate authority's authorization for the recipient to perform a task. The authorization may be the authorization to access a given datum. The authorization may be the authorization to access a given process. In some embodiments, the certificate may identify the certificate authority. The digital certificate may include a digital signature.

With continued reference to FIG. 1E, in some embodiments, a third party such as a certificate authority (CA) is available to verify that the possessor of the private key is a particular entity; thus, if the certificate authority may be trusted, and the private key has not been stolen, the ability of an entity to produce a digital signature confirms the identity of the entity and links the file to the entity in a verifiable way. Digital signature may be incorporated in a digital certificate, which is a document authenticating the entity possessing the private key by authority of the issuing certificate authority and signed with a digital signature created with that private key and a mathematical representation of the remainder of the certificate. In other embodiments, digital signature is verified by comparing the digital signature to one known to have been created by the entity that purportedly signed the digital signature; for instance, if the public key that decrypts the known signature also decrypts the digital signature, the digital signature may be considered verified. Digital signature may also be used to verify that the file has not been altered since the formation of the digital signature.

With continued reference to FIG. 1E, in some cases, processing circuit 150 may perform one or more signal processing steps on a signal. For instance, apparatus 100 may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIG. 1E, in some cases, As used in this disclosure, a “power source” is a component that stores energy. In an embodiment, power source 152 may include a battery component that stores energy in a chemical form and releases it as electrical energy to power other components of apparatus 100 e.g., aerosol delivery mechanism 108. In a non-limiting example, power source 152 may include a plurality of batteries. Battery may include an anode (i.e., negative electrode), a cathode (i.e., positive electrode), an electrolyte, and a separator. In some cases, power source 152 may include, without limitation, Lithium-ion (Li-ion) battery, Nickel-Cadmium (NiCd) battery, Nickel-Metal Hydride (NiMH) battery, Alkaline battery, Lead-Acid battery, or the like. In some cases, at least a battery component may come in various shapes and sizes such as, without limitation, cylindrical cells (e.g., AA), flat pouch cells, prismatic cells and/or the like.

With continued reference to FIG. 1E, in some cases, power source 152 within device 144 may include a protection mechanism. For instance, and without limitation, modern batteries, such as Li-ion battery, may come with built-in protection circuits to prevent overcharging, over-discharging, and overheating, and/or the like to ensure safe operation of apparatus 100. In a non-limiting example, protection circuit may utilize one or more sensors as described herein such as a temperature sensor to monitor battery's internal temperature and cut off power supply if it exceeds a certain threshold. In some cases, power source 152 may be disposed proximal to the end of device connected to cartridge 102 such that positive terminal 156a and negative terminal 156b may be in contact with electrical interface 118 directly or through conductive path i.e., wires as described above, to enable energy (electrical power) transfer. In a non-limiting example, spring-loaded contacts may be used to ensure a consistent and secure connection to positive and negative terminals 156a-b as described above.

With continued reference to FIG. 1E. in a non-limiting example, at least one battery component (i.e., power source 152) within second compartment 148b may also be eco-friendly by implementing biodegradable electrolytes, as well as replacing non-biodegradable, petroleum-based polymers with those that can easily degrade, thereby minimizing the usage of non-renewable resources in power source 152. By removing metals, using biodegradable polymers, and implementing biodegradable electrolytes, batteries become biodegradable themselves. However, even if power source 152 is a lithium-ion battery, a fully biodegradable plastic construction can allow the user to take out the battery of the device, recharge it, and reinsert it into a new device while composting or disposing of the old device. In a non-limiting example, device 144 may include a battery holder that is insertable into housing 146 and protects the user from handling a battery directly. In this case, a button or pair of buttons can be affixed to the outer shell to allow the user to press and take the battery out, recharge it, or dispose of it. Reinserting the charged or new battery can then power the device again, and the electrical connection can be formed with a plurality of contacts, such as long contacts as show in 120a-b, dome switches, leaf spring contacts, a pair of pins or another method just as when forming an electrical connection with cartridge 102 upon insertion may be employed by apparatus 100.

With continued reference to FIG. 1E, exemplary embodiments of battery component may include, without limitations, removable batteries, integrated batteries, flexible batteries, thin film batteries, among others. As an ordinary person skilled in the art, upon reviewing the entirety of this disclosure, will be aware of various types of battery suitable for different configurations depending on design requirements and power consumption of apparatus 100 as described herein. It should be noted that the choice of battery may be optimized for longevity, charge cycles, weight, form factors, and/or the like.

With continued reference to FIG. 1E, in some cases, a second set of resistors 158 may be connected with power source 152 to serve as a voltage divider or a current limiter. In some cases, second set of resistors 158 may be configured to stabilize the voltage levels, preventing potential spikes or drops that may harm other electronic components within device 144. In a non-limiting example, second set of resistors 158 may be configured to ensure battery discharges at a safe and consistent discharge rate. In a non-limiting example, second set of resistors 158 may be placed close to positive and negative terminals 156a-b, in between a charging circuit 160 (i.e., an electronic circuit designed to charge power source 152 in case of power source 152 being rechargeable) and power source 152. In some cases, charging circuit 160 may be configured to provide a voltage input for other electronics of apparatus 100. Additionally, or alternatively, second set of resistors 158 may be used in conjunction with other components to provide feedback to charging circuit 160, for example, and without limitation, charging rate may be adjusted by charging circuit when the battery is full. In other cases, if battery component is accidentally connected backward, second set of resistors 158 may be configured to prevent a short circuit.

With continued reference to FIG. 1E, device may include at least a sensor 162. At least a sensor 162 may be communicatively connected to processing circuit 150. In some cases, at least a sensor 162 may be configured to detect and monitor specific data related to device's (both internal and external) operations or surrounding environment (including user). In some cases, at least a sensor 162 may include a temperature sensor configured to detect temperature as device 144 operates. In some cases, at least a sensor 162 may include an electrical sensor configured to monitor electrical conductivity (e.g., voltage level or current level) or resistance within device 144. In some cases, at least a sensor may include a pressure sensor. In some embodiments, pressure sensor may be configured to detect changes in internal pressure within device 144. Such changes could be indicative of various events or conditions e.g., a malfunction, a leak, among others. In some cases, pressure sensor may be configured to monitor user interaction. For instance, and without limitation, a change in internal or external pressure may indicate that the user has picked up the apparatus 100. Conversely, a return to a baseline pressure may suggest that the user has placed the device down. In a non-limiting example, pressure sensor may be employed as a switch, controlling the operation of the device. When the pressure sensor detects a pressure above a certain threshold (indicative of user interaction), device may be activated, preparing it for use. Once the pressure drops below this threshold, the device may be automatically switched off or enter a standby mode. In other cases, a gradual increase in pressure may indicate inhalation. In an embodiment, processing circuit 150 may be configured to adjust device output (of aerosol) based on the intensity and duration of the inhalation.

With continued reference to FIG. 1E, additionally, or alternatively, at least a sensor 162 may include a biometric sensor. As used in this disclosure, a “biometric sensor” is a device that captures and measures specific physiological or behavioral characteristics of the user for biometric identification or authentication. In an embodiment, biometrics may include unique and measurable traits of the user which may be used to verify user's identity and grant access to apparatus 100. In a non-limiting example, biometric sensor may include any device that integrates fingerprint scanner, facial recognition solution, voice recognition, iris scans, palm prints, hand geometry, and/or the like to limit only authorized users from using apparatus 100 for the delivery for aerosolizable material 104 delivery and/or aerosol generation. In some cases, apparatus 100 described herein may be activated at the point of sale, after verifying user ID, a limited time window to fingerprint user on apparatus 100 may be given to the authorized purchaser (in some cases, authorized purchaser may be the user); apparatus 100 may need to be reactivated at a point of sale to limit aftermarket sale. However, user within a specific amount of time uses a finger, for example, and without limitation, a thumb on their hand of use, biometric sensor such as a finger printer scanner may be allowed to take shots from a few angles. In a non-limiting example, fingerprint scanner may be first activated (e.g., turned on) prior to the activation of the device through a wireless communication device upon receiving an activation datum from an external device in communication with the wireless communication device as described in further detail below with reference to FIG. 1F. Such fingerprint scan may then be used to reactivate apparatus 100 (either per inhalation, or for a specific amount of time) for the authorized user later. Biometrics data may be encrypted according to methods described in a later section. Biometrics user data for the purpose of youth access prevention is also subject to biometric data regulation, such as for example 740 ILCS 14/Biometric Information Privacy Act (BIPA). These regulations typically require private entities in possession of biometric identifiers or biometric information to develop a written policy establishing a retention schedule and guidelines for permanently destroying biometric identifiers and biometric information when the initial purpose for collecting. Importantly, biometric information may not be uploaded into the cloud but remain locally on the device and initiate a data wipe at a pre-specified time such as 6 months or a year.

With continued reference to FIG. 1E, other exemplary embodiments of sensor 162 may include, without limitation, humidity sensor, optical sensor, gyroscope, and the like. As a person skilled in the art, upon reviewing the entirety of this disclosure, will be aware of various sensors that may be integrated into device 144 to enhance device's capabilities and user experience. It should be noted that sensors described herein may be singular in their function or may be combined in sensor suit (i.e., a combination of multiple sensors working in tandem). In a non-limiting example, at least a sensor 162 or a sensor suit may be placed on compartment tray 154 at a strategic location i.e., in close proximity to components they monitor or the housing top 145a, allowing for real-time feedback. In some cases, compartment tray 154 may include a dedicated sensor slot 164 or a molded cavity, wherein at least a sensor 162 or sensor suit may be securely positioned.

With continued reference to FIG. 1E, in some cases, compartment tray 154 may further include a plurality of indicator slots 166 configured to securely house and position a plurality of indicators 168. In some embodiments, plurality of indicators 168 may serve as a visual representation of various operational states, conditions, or alerts related to the device's functionality and performance. In some cases, at least a portion of housing top 146a may be transparent and/or hollow such that user may visually inspect plurality of indicators 168 or other internal components and workings of the device. In a non-limiting example, a plurality of LED indicators or a display (e.g., OLED or LCD screen) may be integrated into indicator slots 166. In some cases, LED indicators may be of different colors, each representing a specific state or alert. In some cases, a green LED might indicate that the device is fully charged or in optimal working condition, a red LED may signify a low battery or a malfunction that requires user attention, a blue LED may represent a Bluetooth or wireless connection status, and a blinking yellow LED may indicate that the device is in a standby or sleep mode.

With continued reference to FIG. 1E, in some cases, display may provide more detailed information, such as, without limitation, battery percentage, temperature readings, or even user-specific messages. In some embodiments, behaviors of plurality of indicators 168 may be directly influenced by data received from one or more sensors as described herein. For example, and without limitation, if temperature sensor detects an overheating condition, at least one indicator of plurality of indicators 168 may flash red rapidly to alert the user. For another example, a timed countdown to self-verify fingerprint on fingerprint scanner after unlocking of apparatus 100 electrical system (i.e., device activated) may be displayed. For another example, a pressure sensor detecting a sudden change (e.g., device being picked up or put down) may trigger at least one indicator of plurality of indicators 168 to illuminate in a specific pattern or display message indicating “Active” or “Inactive” status. In other cases, plurality of indicators may respond to user's inhalation. For example, and without limitation, LED light may glow brighter with a strong inhalation, providing visual feedback to the user.

Now referring to FIG. 1F, a block diagram of an exemplary embodiment of apparatus 100 is illustrated. Apparatus 100 may be powered by power source 152 e.g., a 3.7V, 350 mAh battery. In some cases, such battery may be rechargeable. Processing circuit 150 may be connected with charging circuit 160, wherein charging circuit 160 may be connected with power source 152, configured to charge the battery. In a non-limiting example, apparatus 100 may be charged through a Micro USB cable, Power source 152, processing circuit 150, charging circuit 160 may be mounted inside device 144, as described above with reference to FIGS. 1A-E.

With continued reference to FIG. 1F, processing circuit 150 within device 144 is configured to detect a sensed datum 170 pertaining to a user using at least a sensor 162 as described above. As used in this disclosure, a “sensed datum” is any piece of information or data that is captured, measured, or detected by one or more sensors in real-time or near-real-time. Sensor may include any sensor described herein such as, without limitation, pressure sensor, biometric sensor, temperature sensor, electrical sensor, and the like. In some cases, sensed datum 170 may include, without limitation, physical parameters, environmental conditions, user interactions, or any other measurable attribute. In some cases, sensed datum 170 may include any combination of data collected from one or more sensors or sensor suites. In some embodiments, sensed datum 170 may be processed by processing circuit 150 for various purposes, including youth access prevention, device authentication, safety measurement, device performance optimization, overall user experience enhancement, and the like as described in detail below.

With continued reference to FIG. 1F, in a non-limiting example, sensed datum 170 may include user behavior data. As used in this disclosure, “user behavior data” is a collection of information or data points that capture and describe one or more actions, patterns, preferences and habits of users when interacting with the device. In some cases, user behavior data may be related to the airflow or pressure changes within the device, in which a pressure sensor may be configured to detect variations when a user interacts with an inhale port 172 (e.g., airflow tube or vapor tube as described above). In a non-limiting example, user behavior data may assist in conducting clinical studies on relative effectiveness of device in switching smokers to the product and include a data point describing a strength and/or a duration of the user's inhalation. In another non-limiting example for youth access prevention, sensed datum 170 pertaining to the user may also include user biometric data such as fingerprint patterns, facial features, voice patterns/tones/frequency, retinal or iris scans, breathing patterns, behavioral biometrics and/or the like.

With continued reference to FIG. 1F, in some cases, plurality of indicators 168 may be manipulated by processing circuit 150 based on such user behavior data. Indicators may include any indicators as described above such as, without limitation, one or more LED indicators. LED indicators may be connected to one or more General-Purpose input/output (GPIO) pins on processing circuit 150. One or more MOSFETs may be used to switch LED indicators on and off. In some cases, if user behavior data indicates frequent usage of device within short time interval, at least one of the indicators 168 may flash a warning light suggesting user take a break or if user wishes to lock themselves out through an app or webapp, indicate that the device is not yet usable. In some cases, processing circuit 150 may be configured to illuminate at least one of the indictors 168 to show a particular device mode is active. In other cases, when user behavior data suggests an unusual or potentially harmful pattern e.g., failure in biometric data verification, processing circuit 150 may activate at least one indicator to emit a red light.

With continued reference to FIG. 1F, processing circuit 150 is configured to read a unique identifier 174 associated with resistor 128 upon an electrical connection 178 of cartridge 102 and device 144 through electrical interface 118 as described above. As used in this disclosure, an “unique identifier” is a specific value or set of values that is distinct and serves to distinguish one entity from all other entities. In an embodiment, unique identifier may include a sequence of numbers. In another embodiments, unique identifier may include a combination of numbers, letters, and/or characters. In a non-limiting example, unique identifier 174 may include a resistance value or a combination of a plurality of resistance values associated with a plurality of resistors 128 read by resistance reading circuit 176 (as described above). A known voltage or current may be applied by processing circuit 150 to each resistor in the set (one at a time) a resulting current or voltage may be measured (respectively). In some cases, Ohm's law: V=I·R may be used to determine the resistance. In some cases, processing circuit 150 may be configured to convert measured resistance value into a digital format. An ADC may be used to translate analog resistance measurement into digital values. Digital values corresponding to each resistor's resistance may be combined, by processing circuit 150 in a specific sequence to form unique identifier 174. In an embodiment, this combination may include a straightforward concatenation, or it may involve more complex encoding/encryption schemes as described above.

With continued reference to FIG. 1F, in some cases, unique identifier 174 may include an element of data that uniquely identifies any components within apparatus 100 such as, without limitation, power source 152, charging circuit 160, second set of resistors 158, at least a sensor 162, and/or any combination thereof. In a non-limiting example, a unique identifier associated with a wireless communication device e.g., an NFC chip as described in detail below may be read by processing circuit 150 for user authentication and/or device pairing purposes. In another non-limiting example, unique identifier 174 may be derived from a specific (electrical) pin layout (i.e., a combination of active [connected] pins and inactive [disconnected or missing] pins). In such embodiment, processing circuit 150 may be configured to detect the presence or absence of each pin in the set, creating a binary pattern; for instance, the presence of a pin may be represented by “1” and its absence by “0.” Additionally, or alternatively, unique identifier 174 may include any unique identifiers as described in U.S. patent application Ser. No. 18/211,726 (Attorney docket number 1445-002USU1), filed on Jun. 20, 2023, and entitled “APPARATUS AND METHOD FOR UNIQUE IDENTIFICATION OF AN OBJECT USING NEAR-FIELD COMMUNICATION (NFC),” which its entirety is incorporated herein by reference.

With continued reference to FIG. 1F, processing circuit 150 is configured to validate sensed datum 170 and unique identifier 174. As used in this disclosure, “validation” is a process of ensuring that which is being “validated” complies with stakeholder expectations and/or desires. Stakeholders may include users, device manufacturer, property owners, regulators, customers, and the like. Very often a specification prescribes certain testable conditions (e.g., sensed datum 170 and unique identifier 174) that codify relevant stakeholder expectations and/or desires. In some cases, validation includes comparing a product, for example without limitation sensed datum 170 and unique identifier 174 against a specification. Such validation process may ensure that sensed datum 170 and unique identifier 174 are genuine and correspond to known and approved values or configurations.

With continued reference to FIG. 1F, in some cases, validating sensed datum 170 and unique identifier 174 may involve a series of checks and verifications. One such check may include, without limitation, recognizing the cartridge 102 based on its unique identifier 174. In some cases, recognition of cartridge 102 may be primarily achieved by examining resistance value associated with resistor 128 as described above. Processing circuit 150 may be configured to cross-references unique identifier 174 with a database 180 containing a list of approved unique identifiers. “Cross-referencing,” for the purpose of this disclosure, means comparing the obtained unique identifier 174 with entries in the database 180 to find a match or similarity. In a non-limiting example, if unique identifier 174 matches an entry in the database, processing circuit 150 may confirm the authenticity of cartridge 102. In some cases, database 180 may contain unique identifiers for various types of cartridges, each associated with specific set of characteristics e.g., flavor, nicotine content, brand, among others. For instance, and without limitation, different cartridges may contain aerosolizable materials in different flavors, wherein unique identifier 174 may help determine which flavor cartridge 102 contains. In some cases, if the cartridge's unique identifier 174 does not match any entry in database 180, it may be flagged as unauthenticated, potentially indicating a counterfeit or unauthorized product.

With continued reference to FIG. 1F, in a non-limiting example, processing circuit 150 may be configured to utilize a lookup table (LUT), at least in part, to cross-reference unique identifier 174. A “lookup table,” for the purpose of this disclosure, is a data structure, (in some cases, an array or associative array) used to replace a runtime computation with a simpler array indexing operation. For example, LUT may include an array of data that maps input values to output values. In some cases, LUT may be stored in memory as described above. In some cases, saving in terms of processing time may be significant, since retrieving a value e.g., an approved unique identifier from memory (i.e., array indexing operation) is faster than undergoing an “expensive” computation (e.g., querying a database 180) or I/O operation. In some embodiments, LUT may include a pre-populated list of approved unique identifiers, wherein each data entry in LUT may correspond to an approved unique identifier. In some cases, additional information related to that identifier such as, without limitation, manufacturing date, batch number, or other relevant metadata may also be included. Upon receiving unique identifier 174, processing circuit 150 may search LUT for such unique identifier. If found, unique identifier 174 may be a valid unique identifier or additional information may be retrieved for additional verification, logging, and/or user notification purposes. Conversely, if unique identifier 174 is not found in LUT, processing circuit 150 may be configured to execute appropriate processes such as, without limitation, denying certain functionalities, alerting the user, suggesting further steps, and/or the like.

With continued reference to FIG. 1F, in some cases, processing circuit may additionally, or alternatively include a wireless communication device 182 configured to communicate sensed datum 170 and unique identifier 174 to an external device 184. As used in this disclosure, a “wireless communication device” is a device that is capable of communicating with other devices e.g., external device 184 without a physical and electrical connection. As used in this disclosure, an “external device” is any device exterior to apparatus 100 that communicates with elements within apparatus 100. In some embodiments, external device 184 may include any additional computing device, such as a mobile device, laptop, desktop computer, or the like. In a non-limiting example, external device 184 may include a transceiver (i.e., a combination of transmitter and/or receiver in a single package) configured to transmit, as well as receive signals. In another non-limiting example, external device 184 may include an NFC reader. In some cases, validating sensed datum 170 and unique identifier 174 may include communicating, using wireless communication device 182, sensed datum 170 and unique identifier 174 to external device 184 for a remote validation. In some cases, external device 184 may include a server configured to generate an activation datum based on the remove validation. As used in this disclosure, an “activation datum” is a specific piece of data or set of data points used to activate or trigger a particular function or process in a device or system. For instance, activation datum may include a validation datum as described in U.S. patent application Ser. No. 18/211,706 (Attorney docket number 1445-001USU1), filed on Jun. 20, 2023, and entitled “APPARATUS AND METHOD FOR AEROSOL DELIVERY,” which its entirety is incorporated herein by reference. In other cases, device 144 may directly include an NFC reader; for instance, and without limitation, NFC reader may be installed in the base of the device 144 receptable (e.g., bottom of the third compartment 148c). Such in-device NFC reader may allow locking/unlocking cartridge 102 when cartridge 102 is attached to device 144 instead of at point of sale or manufacture.

With continued reference to FIG. 1F, in some cases, communication between wireless communication device 182 and external device 184 may include the use of Bluetooth Low Energy (Bluetooth LE, colloquially BLE) as a wireless personal area network technology. Such technologies may be combined with the NFC-enabled technology as described below to provide youth access prevention features and user setting optimization with end-user having the ability to control settings and systems of devices such as processing circuit 150 within apparatus 100 via one or more software applications (i.e., computer programs); for instance, and without limitation, plurality of device settings may be customized by user through a mobile app.

With continued reference to FIG. 1F, additionally, or alternatively, apparatus 100 as described herein may be paired with a digital therapeutic or video counseling application for behavioral counseling of the user, enhancing user's health and wellness. In some cases, the integration of digital therapeutic or video counseling application may leverage the capabilities of pod recognition and NFC/BLE technology to connect apparatus 100 with an installed digital therapeutic software (on remote user device). In an embodiment, processing circuit 150 may be configured to initiate a communication with the digital therapeutic application based on pod recognition as described in further detail below. In some cases, usage data from apparatus 100 may be transmitted to the application, enabling real-time monitoring and personalized feedback. In a non-limiting example, physicians may be able to access such data for analytic and diagnostic purposes, potentially aiding in customization of treatment plans and monitoring patient progress. User may access video counseling sessions (i.e., face-to-face interactions) through said application, connecting with one or more healthcare professionals for real-time support and guidance. In some cases, video counseling may be scheduled through the application. Further, to encourage consistent use and positive behavior changes, said application may include an implementation of an incentive system. In one or more embodiments, users may earn rewards or points for meeting certain goals or milestones e.g., reduced usage or consistent engagement with counseling sessions.

With continued reference to FIG. 1F, in some cases, wireless communication device 182 may be configured to communicate with external device 184 within a communication network such as, without limitation, a wide area network (e.g., the Internet, an enterprise network), a local area network (e.g., a network associated with an office, a building, a campus or other relatively small geographic space), a telephone network, a data network associated with a telephone/voice provider (e.g., a mobile communication provider data and/or voice network), a direct connection between two computing devices, and any combination thereof.

With continued reference to FIG. 1F, additionally, or alternatively, wireless communication device 182 may use radio frequency identification (RFID) to communicate with external device 184, wherein the RFID is a form of wireless communication that incorporates the use of electromagnetic or electrostatic coupling in the radio frequency portion of the electromagnetic spectrum to uniquely identify an object such as, without limitation, apparatus 100. In some embodiments, wireless communication device 182 using RFID may include a transponder, wherein the transponder is a component that configured to respond to different incoming signals. Further wireless communication device using RFID may be operate under different frequency; for instance, wireless communication device may operate at various frequency including, without limitation, low frequency (30 KHz to 700 KHz), high frequency (3 MHz to 30 MHz), Ultra high frequency (300 MHz to 960 MHz), and the like. In a non-limiting example, cartridge 102 and/or device 144 may include an embedded RFID tag having a unique identifier. In some cases, cartridge 102 and/or device 144 may remain in a ‘locked’ state until activated by a (retail) beacon (i.e., external device 184) installed at point of sale or exit points in a retail environment that is configured to communicate with RFID tag and sends an unlocking command upon confirmation of a legal sale. Unlocking commands can be received in different ways, for instance by tapping a device on a reader or by providing an unlock command for the specific ID of the product after it is sold, by for instance scanning the packaging unique QR code or bar code, or by integrating the point of sale system of the retail store to sync sales records with the retail beacon.

With continued reference to FIG. 1F, as used in this disclosure, a “near field communication chip” is a component that enables processing circuit 150 to communicate with devices such as external device 184 wirelessly, within a short range using near-field communication technology, wherein the near-field communication technology may enable NFC chip to execute a plurality of communication protocols that enables communication between two devices, such as, without limitation, wireless communication device 182 to external device 184, over a distance of 4 cm (1.5 inches) or less. NFC chip may offer a low-speed connection used to bootstrap one or more wireless connection similar to proximity card technology; for instance, and without limitation, NFC chip may function as a smart card.

With continued reference to FIG. 1F, in some cases, wireless communication device 182 may further include one or more antenna may be connected to NFC chip. As used in this disclosure, an “antenna” is a device configured to convert voltage from a transmitter into a radio signal. Antenna may pick radio signals out of the air and convert them into voltage for recovery in a receiver. In an embodiment, antenna may include a transducer. In a non-limiting example, wireless communication device 182 with NFC chip connecting to two antennas may communicate with external device 184 in both directions using a frequency of 13.56 MHZ in globally available unlicensed radio frequency ISM band using ISO/IEC 18200-3 air interface standard at data rates ranging from 106 to 424 kbit/s. In some cases, NFC chip may be disposed externally to device 144. In a non-limiting example, NFC chip may include an NFC sticker that adheres to the exterior of device casing. Wireless communication device incorporating NFC technology is described in greater detail in U.S. patent application Ser. No. 18/211,706.

With continued reference to FIG. 1F, in some cases, validating sensed datum 170 and unique identifier 174 may also include comparing sensed datum 170 against a user profile 186 pertaining to the user. In some cases, comparison of sensed datum 170 with user profile 186 specific to the user in question may ensure cartridge 102 and/or device 144 not only matches product's specifications, but also aligns with user's identify, preferences, habits, historical data and the like. As used in this disclosure, a “user profile” is a comprehensive collection of data and information related to a specific user for the purpose of youth access prevention. In some cases, user profile 186 may include a plurality of user metadata such as, without limitation, user's preferences, usage habits, purchase history, feedback, biometric data, and any other relevant information that can provide insights into the user's behavior and preferences. In some cases, described user profile 186 may be created locally, by processing circuit 150, based on sensed datum 170 e.g., data collected from user's interactions with the device, feedback, and other data sources as described herein. Such data may evolve over time as more data is accumulated. In other cases, user profile 186 may be generated remotely, by external device 184 as described above, as a function of sensed datum 170 (and unique identifier 174) received from wireless communication device 184. Additionally, or alternatively, user profile 186 may be generated by associating user behavior data with unique identifier 174. In some cases, user profile 186 may be stored in database 180. Comparing sensed datum 170 against user profile 186 may include retrieving, based on associated unique identifier 174, user profile 186 from database 180. Further, in some cases, comparing sensed datum 170 with user profile 186 may determine a cartridge suitability for the user. For instance, and without limitation, if user profile 186 indicates a preference for a step down program of nicotine strength, and sensed datum 170 from cartridge 102 doesn't match this preference, processing circuit 150 may flag this discrepancy i.e., an indication of an incorrect cartridge 102 placement or a potential cartridge mismatch that needs addressing.

With continued reference to FIG. 1F, additionally, or alternatively, validating sensed datum 170 and unique identifier 174 may further requiring user to provide two distinct forms of identification before access is granted to add extra layer of security. In some cases, first form of identification may include sensed datum 170 and/or unique identifier 174 as described herein (i.e., something the user has). In some cases, processing circuit 150 may require “something the user knows,” for instance, and without limitation, a pin, password, a pattern, a verification code, an answer to a security question, among others. When user attempts to access the apparatus, subsequent to the attachment of pod 102, user may be required to first enter at least one second form of identification. In some cases, the at least one second from of identification may be enter via a remote user device (for example though a software application) that is paired with apparatus 100.

With continued reference to FIG. 1F, in case of sensed datum 170 and unique identifier 174 validation is performed remotely, processing circuit 150 may be configured to receive an external response from external device. In some cases, external response may be generated as a function of a request from wireless communication device 182, such as, without limitation, request for sensed datum 170 and unique identifier 174 verification. In some cases, such external response may be generated by one or more web APIs. For instance, and without limitation, external device may include, or may be communicatively connected to a remote server, wherein the remote server may implement one or more APIs configured to process, analyze, and/or verify sensed datum 170 and unique identifier 174. In some cases, validating sensed datum 170 may include comparing sensed datum 170 with a historical sensed datum 170, wherein the historical sensed datum 170 may include pre-saved testing data of apparatus 100 at the point of manufacture or initial settings at point of sale. In a non-limiting example, processing circuit 150 may be configured to perform age restriction on the use of the device. In some cases, generated user profile 186 may be required to contain user metadata specifying user's age is over 21.

With continued reference to FIG. 1F, processing circuit 150 is configured to activate aerosol delivery mechanism 108 of cartridge 102 as a function of a positive validation of sensed datum 170 and unique identifier 174. In some cases, an authentication datum 188 may then be generated upon a successful match of both unique identifier 174 and sensed datum 170. As used in this disclosure, an “authentication datum” is a piece of data or a set of data that acts as a digital “seal of approval” or a confirmation that device and components thereof have been authenticated. In some cases, authentication datum 188 may be generated by processing circuit 150 or external device 184 in a form of response to data validation. In some cases, authentication datum 188 may include, but is not limited to, a notification, a digital certificate, a unique code, a token, or any other form of digital confirmation. In some cases, generated authentication datum 188 may be displayed to user, stored for future reference, or used to unlock device functions and/or additional features of In some cases, authentication datum may be configured as a proof that the apparatus 100 been successfully authenticated.

With continued reference to FIG. 1F, in a non-limiting example, authentication datum 188 may include any means configured to modify an internal state of processing circuit 150 based on validation of sensed datum 170 and unique identifier 174. For instance, and without limitation, processing circuit 150 may change internal operation state of device according to authentication datum 188. As used in this disclosure, an “internal state” is a value representing an internal property, attribute, or otherwise a status of processing circuit 150. In some cases, processing circuit 150 may switch internal state from a first state i.e., inactive (lock) to a second state i.e., active (unlock). In a non-limiting example, processing circuit 150 may be configured to activate aerosol delivery mechanism 108 of cartridge 102 may include activating aerosol delivery mechanism 108 as a function of authentication datum 188. In a non-limiting example, authentication datum 188 may include at least a command or an instruction set that can modify internal state of processing circuit 150 based on successful validation of sensed datum 170 and unique identifier 174. Once device is authenticated, it is ready to deliver aerosol to the user.

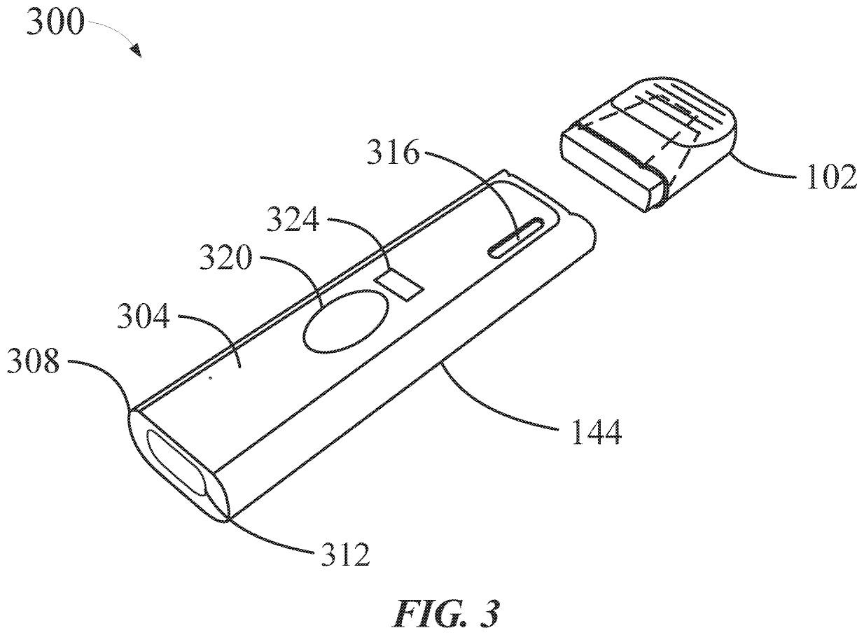

With continued reference to FIG. 1F, in other cases, authentication datum 188 may include a token (or a token certificate), wherein the “token,” for the purpose of this disclosure, is a digital representation of authentication and authorization data. In some cases, token may encapsulate one or more pieces of information, e.g., user identity, device identity, session details, and the like. In an embodiment, upon a successful validation of sensed datum 170 and unique identifier 174, processing circuit 150 and/or external device 184 may be configured to generate at least one token. In a non-limiting example, at least one token may be used to manage user sessions, ensuring user doesn't need to re-authenticate for every operation during a session. Additionally, or alternatively, at least one token may include an encryption key as described above. Further, in some cases, at least one token may include a Non-Fungible Token (NFT). As used in this disclosure, an “NFT” are unique digital assets verified using blockchain technology as described in detail below with reference to FIG. 3 In an embodiment, NFT may include a digital badge, signifying ownership of device and that the user or the device has been authenticated. At least one token may be pushed on an immutable sequential listing. As used in this disclosure, an “immutable sequential listing” is a data structure that places data entries in a fixed sequential arrangement, such as a temporal sequence of entries and/or blocks thereof, where the sequential arrangement, once established, cannot be altered or reordered. An immutable sequential listing may be, include and/or implement an immutable ledger, where data entries that have been posted to the immutable sequential listing cannot be altered.

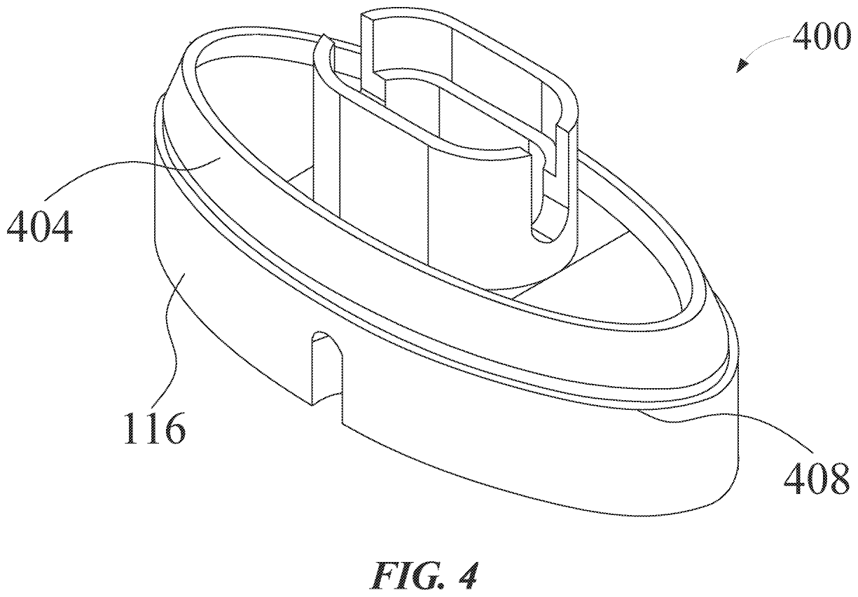

With continued reference to FIG. 1F, additionally, or alternatively, validating sensed datum 170 and unique identifier 174 may include utilizing one or more machine learning processes. In some cases, authentication datum 188 may be generated using one or more machine learning models generated using machine learning module as described in detail below with reference to FIG. 4. In some cases, Machine learning module to implement one or more algorithms or generate one or more machine learning models, however, the machine learning module is exemplary and may not be necessary to generate one or more machine learning models and perform any machine learning described herein. In one or more embodiments, one or more machine-learning models may be generated using training data. Training data may include inputs and corresponding predetermined outputs so that a machine-learning model may use correlations between the provided exemplary inputs and outputs to develop an algorithm and/or relationship that then allows machine-learning model to determine its own outputs for inputs. Training data may contain correlations that a machine-learning process may use to model relationships between two or more categories of data elements. Exemplary inputs and outputs may come from database 180 as described herein or any other databases, or even be provided by the user. In a non-limiting example, machine-learning module may obtain a training set by querying database 180 that includes past inputs and outputs. Training data may include inputs from various types of databases, resources, and/or user inputs and outputs correlated to each of those inputs so that a machine-learning model may determine an output. Correlations may indicate causative and/or predictive links between data, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements. In a further embodiment, training data may include previous outputs such that one or more machine learning models iteratively produces outputs.

With continued reference to FIG. 1F, in a non-limiting example, processing circuit 150 and/or external device 184 may be configured to generate a device authentication machine learning model using authentication training data, wherein authentication training data may include a plurality of unique identifier as input correlated to a plurality of authentication datum as output. In some cases, authentication training data may include a plurality of sensed datums as input correlated to a plurality of authentication datum as output. In some cases, more than one authentication datums may be generated, for example, and without limitation, a first authentication datum may be generated as a function of unique identifier 174 using trained device authentication machine learning model and while a second authentication datum may be generated based on sensed datum 170. In such an embodiment, authentication datum 188 may be generated by combining the first authentication datum and the second authentication datum. In some cases, combining multiple authentication datums may include applying Boolean logic (e.g., “AND,” “OR,” “NOT,” and the like) to each individual authentication datum.

With continued reference to FIG. 1F, in some embodiments, generating authentication datum 188 may include determining a device usability. As used in this disclosure, a “device usability” refers to a degree to which user may use apparatus 100's functions as described herein; for instance, and without limitation, vaping using apparatus 100. In some embodiments, device usability 168 may include what functionalities of apparatus 100 user may use and/or may not use. In some cases, functionalities of apparatus 100 may include, without limitation, powering on/off, initiating/terminating vaporization of aerosolizable material, configuring aerosol delivery mechanism (i.e., adjusting temperature), changing aerosolizable material, and the like thereof. In a non-limiting example, when cartridge 102 is attached to device 144, compatibility and authenticity of cartridge 102 (and associated unique identifier) may be verified. If cartridge 102 is recognized as authentic and compatible (i.e., a match of unique identifier), and user biometric data aligns with stored user profiles associated with authorized user, full access to the device's functionalities may be granted. Conversely, if cartridge 102 is deemed incompatible or counterfeit (i.e., a mismatch of unique identifier) or user's biometric data fails to authenticate, certain functionalities, such as adjusting temperature or vaporization initiation, may be restricted, or disabled.

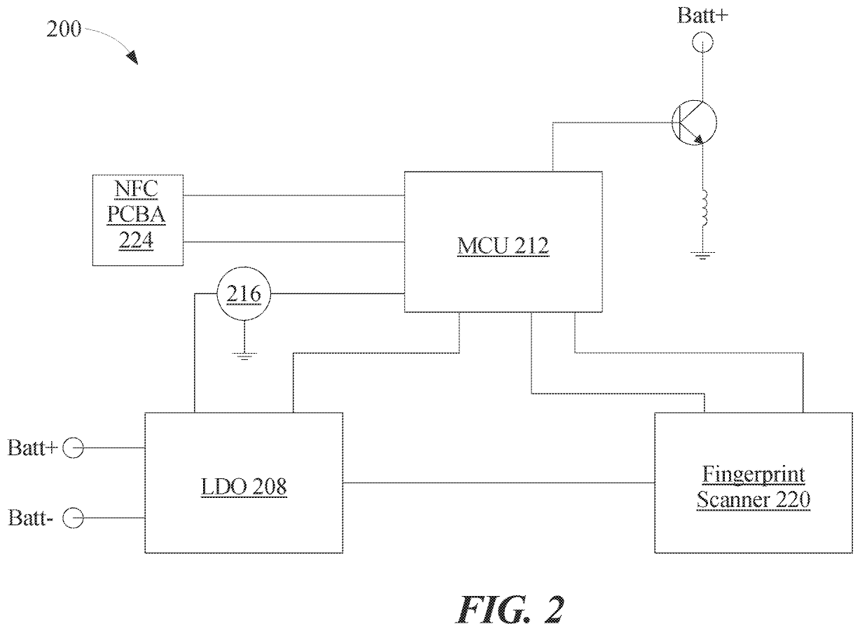

With continued reference to FIG. 2, a schematic of an exemplary embodiment of a device circuitry 200 is illustrated. Device circuitry 200 may integrate a battery 204, a Low Dropout Regulator (LDO) 208, a microcontroller unit (MCU) 212, a microphone 216, a fingerprint scanner 220, a NFC PCBA 224, and the like. Device circuitry 200 may be powered by battery 204, which may include one or more positive (Batt+) terminal and one or more negative (Batt-) terminals. LDO 208 may be connected to battery 204 configured to regulate the voltage from battery 204 to a stable level suitable for MCU 212 and other sensitive components. In a non-limiting example, LDO 208 may ensure that fluctuations in battery voltage do not affect the performance of device circuitry 200 as described herein. MCU 212 may include any processing circuit, processor, or computing device as described in this disclosure. In some cases, MCU 212 may include BLE capabilities for wireless communication as described above with reference to FIGS. 1A-F. MCU 212 may be connected to LDO 208 to receive regulated electrical power. In a non-limiting embodiment, microphone 216 may be included in device circuitry 200, connected between LDO 208 and MCU 212, wherein the microphone 216 may be used for voice recognition or audio input, complementing fingerprint scanner 220 for a multi-factor authentication. In some cases, fingerprint scanner 220 may be connected to MCU 212, wherein the fingerprint scanner 220 may be configured to capture fingerprint data pertaining to a user and send the captured fingerprint data to MCU 212 for processing and authentication as described above with reference to FIGS. 1A-F. NFC PCBA 224 configured for data transfer and device authentication may be connected to MCU 212. In some cases, NFC PCBA 224 may be configured to communicate with other NFC-enabled devices or systems, for example, and without limitation, external device e.g., a NFC reader. Additionally, or alternatively, a transistor, in particular, a BJT NPN transistor may be included in device circuitry 200, with its base connected to MCU 212. of the emitter of the BJT NPN may be connected to the ground, and the controller is connected to Batt+ through an inductor. In a non-limiting example, such transistor may act as a witch or amplifier, controlled by MCU 212. In some cases, device circuitry 200 may include a Complementary Metal-Oxide-Semiconductor (CMOS)

With continued reference to FIG. 2, in one or more embodiments, NFC PCBA 224 may include two antennas. Antennas may include any antenna described above. In a non-limiting example, NFC PCBA 224 may include a first antenna (i.e., ANTI) and a second antenna (i.e., ANT2), wherein the ANTI may be a 2.4/5 GHz Wi-Fi antenna and the ANT2 may be a 2.4 GHz band antenna which may be used for Wi-Fi, ZigBee, Bluetooth, or RF4CE applications. As persons skilled in the art, upon reviewing the entirety of this disclosure, will be aware of various types of antennas and antennas for other frequencies that may be used by NFC PCBA 224 as described in this disclosure. In some embodiments, a NFC chip may be connected with antennas. In some embodiments, antennas may not be attached to NFC PCBA 224. In some cases, a magnetic insulator may be disposed in between antennas and power source 152 to shield antennas from aluminum on power source 152.

Referring now to FIG. 3, an exemplary embodiment 300 of an outer body 304 of device 144 is illustrated. As used in this disclosure, an “outer body” is a container configured to encapsulate a plurality of internal elements of aerosol delivery device 200 such as, without limitation, any elements, components, and/or devices as described in detail below. In some embodiments, outer body 304 may include a variety of shapes. In some cases, outer body 304 may include a flat cylinder shape. In a non-limiting example, outer body 304 may be designed in a shape comparable to an actual cigarette. In some embodiments, outer body 304 may be detachable from apparatus 100. In a non-limiting example, Outer body 304 may be detachable from device 144. In some cases, pod 102 may be attached to device 144, wherein the outer surface of pod 102 may be designed to align and integrate seamlessly with the surface of outer body 304 of device 144.

With continued reference to FIG. 3, in a non-limiting example, bottom 308 of outer body 304 may include a charging connector 312, wherein the charging connector 312 may include any circuit or circuit element by means of which electric power may be transferred from an external power source to power source 152, as described above. For instance, and without limitation, charging connector 312 may include an inductive charging coil whereby electrical power is transferred to the inductive charging coil using a varying exterior magnetic field supplied by another device or a conductive connection from the device 144 to an exterior device. A non-limiting example of a conductive connection may include two or more charge contacts, which may be constructed of conductive material and accessible from an exterior surface of outer body 304, such as, without limitation, bottom 308. Charge contacts may be in electrical communication with power source 108 inside of outer body 304; charge contact pins may be visible on the exterior of outer body 304. When device 144 is connected to an external power source, charging pins may facilitate electrical communication between the power source 108 inside of device 144 and the external power source. Charging pins may be electrically connected to power source 152 via any suitable connection; for instance, and without limitation, charging pins may contact one or more conductive elements including springs, clips, and/or a printed circuit board (PCB). Charging pins may include male and/or female connectors; for instance, charging pins may include a “plug” that projects from bottom 308 of outer body 304 or may include holes into which a plug or one or more projecting conducting pins may be inserted. Additionally, or alternatively, charging connector 312 on bottom 308 may include a magnetic contact.

With continued reference to FIG. 3, in some embodiments, a status indicator 316 may be disposed on any surface of outer body 304. As used in this disclosure, a “status indicator” is an element that continuously indicates one or more status of apparatus 100. Status of apparatus 100 may include, without limitation, state of power source 152, state of aerosol generation mechanism 108 and the like, as described above. In some embodiments, status indicator 316 may include a passive status indicator, wherein the passive status indicator may be a status indicator 316 with physical configurations on outer body 304 which enables one or more indications of current apparatus state. In a non-limiting example, passive status indicator may be disposed on a surface of outer body 304 with a portion of the surface is transparent and/or hollow. User may observe elements, components, or otherwise devices inside outer body 304 through such portion of the surface (i.e., passive status indicator) to know status of apparatus 100. For instance, and without limitation, status indicator 316 may include a liquid fill level indicator, wherein the liquid fill level indicator may passively allow user to acknowledge the amount of aerosolizable material remaining within aerosolizable material reservoir by disposing liquid fill level indicator on the surface of outer body 304 that right above aerosolizable material reservoir. In other embodiments, status indicator 316 may include an active status indicator, wherein the active status indicator may be a status indicator 316 with electrical configurations inside outer body 304 which enables one or more indications of current apparatus state. In a non-limiting example, active status indicator may include an indicator light located on outer body 304. Indicator light may include any light-emitting electronic component, including without limitation a light-emitting diode (LED). Continuing the non-limiting example, liquid fill level indicator may include a LED configured to indicate a detected liquid fill level of aerosolizable material reservoir by illuminating various color of lights; for instance, and without limitation, liquid fill level indicator may illuminate green light when aerosolizable material reservoir is at full capacity and illuminate red light when aerosolizable material at low capacity. In other embodiments, active status indicator may also indicate, without limitation, a charging status of apparatus 100; for instance, and without limitation, indicator light of active status indicator may emit light while the apparatus 100 is charging, and cease illumination when charging is complete. Indicator light of active status indicator may emit a first color of light while charging is occurring and a second when charging is complete, may blink to indicate charging is currently occurring, or the like. Any suitable pattern of illumination in response to charging status of apparatus 100 may be used. In another non-limiting example, active status indicator may indicate positive or negative validation of sensed datum and unique identifier as described above with reference to FIGS. 1A-F. Indicator light of active status indicator may emit, without limitation, color “green” when the validation is positive, and color “red” when the validation is negative.

With continued reference to FIG. 3, additionally, or alternatively, a biometric reading window 320 may be disposed of outer body 304. As used in this disclosure, a “biometric reading window” is a designated area or surface on outer body 304 of apparatus wherein a biometric sensor such as a fingerprint scanner or a microphone is located or integrated. In a non-limiting example, biometric reading window 320 may be recessed into outer body 304, creating a raised or flush surface. Biometric reading window 320 may enable user to interact with biometric sensor through outer body 304, allowing biometric sensor to capture and measure specifics physiological or behavior characteristics of the user. In some cases, the size of biometric reading window 320 may be sufficient to accommodate the specific biometric sensor being used. For example, and without limitation, fingerprint sensor may require a smaller window than a facial recognition sensor. In some cases, size and/or location of biometric reading window may be determined based on ergonomic requirements for ease of use and comfort during normal operation of apparatus 100. In some cases, the surface of biometric reading window 320 may be smooth and free from any imperfections that might interfere with biometric sensor ability to capture accurate biometric data; for instance, and without limitation, surface of biometric reading window 320 may include an oleophobic coating (applied to the sensor surface to reduce the adhesion of oils, dirt, fingerprints, and/or the like). Additionally, or alternatively, biometric reading window 320 may be incorporated into other functional elements such as, without limitation, a power button, status indicator 316, or the like.

With continued reference to FIG. 3, outer body 304 may further include a digital screen 324. In some cases, digital screen 324 may include any display as described above with reference to FIGS. 1A-F. In an embodiment, digital screen 324 may include a simple LCD (liquid crystal display) or an e-ink display which are energy-efficient. In some cases, digital screen 324 may include a compact size e.g., thin, and small, fitting the sleek design of outer body 304. In some cases, digital screen 324 may include one or more lighting conditions. In a non-limiting example, basic information such as, without limitation, battery level, temperature settings, usage counters may be displayed on digital screen 324. In some cases, digital screen 324 may be visible in direct sunlight. In some cases, digital screen 324 may be turned off when apparatus 100 at idle. In some cases, digital screen 324 may be strategically placed for easy visibility and access by user, as shown in FIG. 3. In a non-limiting example, digital screen 324 may be configured to display a timed countdown (e.g., 10 minutes after being purchased [and activated by the NFC chip]) to self-verify fingerprint on fingerprint scanner at biometric reading window 320.

Now referring to FIG. 4, an exemplary embodiment 400 of bottom housing 116 of pod 102 having a chamfered wall 404 is illustrated. In one or more embodiment, bottom housing 116 may include a chamfered wall 404, wherein the “chamfered wall,” for the purpose of this disclosure, is a specific design feature having an angled or beveled edge on a material. Cartridge 102 may include a bottom housing 116 having a chamfered wall 404 located at a joining edge 408 of bottom housing 116, wherein the chamfered wall 404 is configured to facilitate a laser welding of bottom housing 116 to top housing as described above. As used in this disclosure, a “joining edge” is a specific area or line of a component where it is designed to connect, attach, or align with another component. In a non-limiting example, joining edge 408 of bottom housing 116 may be located at the periphery or boundary of bottom housing upper surface. In some cases, joining edge 408 may come into direct contact with top housing's bottom edge when cartridge 102 is assembled. In some cases, chamfered wall may include a transitional edge between two faces of bottom housing. In some cases, chamfered wall 404 may be crucial for process such as laser/ultrasonic welding involved during pod 102 assembly. In a non-limiting example, instead of a sharp 90-degree corner, a chamfer may provide a sloped or angled cut. In some cases, chamfered wall 404 may be uniform along the entire edge of bottom housing 116. In some cases, chamfered wall may be manufactured at the edges where two parts e.g., the upper surface of bottom housing 116 and reservoir 106 with mouthpiece 114 are meet or joined. Such chamfered wall 404 may enable a stronger and more uniform welding by allowing the laser beam to penetrate the joint area more effectively. In other cases, chamfered wall 404 as described herein may simplify the alignment of reservoir 106 and bottom housing 116. In one or more embodiments, laser welding may reduce the number of seals and leak points. In some cases, leaser welding may make cartridges harder or impossible to refill and is easily integrated into automated line manufacturing apparatus 100 as described herein. Additionally, or alternatively, ultrasonic welding may be used to create the solid-state weld between bottom housing 116 and reservoir 106 through chamfered wall 404 without the need for adhesives or additional connectors.

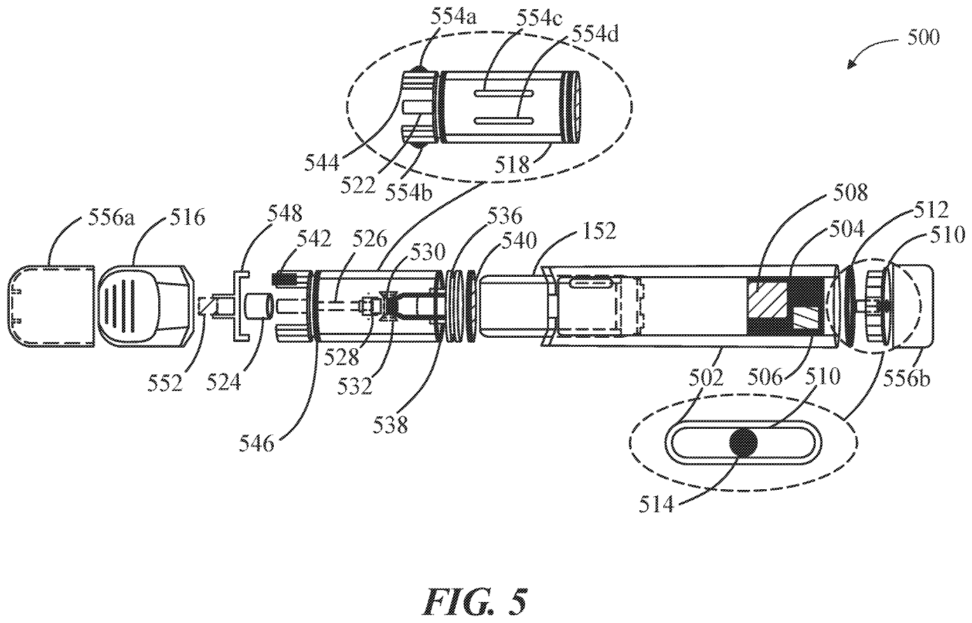

Now referring to FIG. 5, an exemplary embodiment of an aerosol delivery device 500 with cartridge integrated into device is illustrated (in an explosion view). Aerosol delivery device 500 described below may include an aerosol delivery device as disclosed in U.S. patent application Ser. No. 18/511,706. In some cases, aerosol delivery device 500 may include outer body 502. In some embodiments, outer body 502 may be constructed from an injectable mold. In some cases, plastic material such as, without limitation, BIOGRADE B-M (i.e., blend of thermoplastic starch (TPS), aliphatic polyesters (AP) and natural plasticizers (glycerol and sorbitol)) may be injected into the injectable mold under high pressure, filling the space and taking on the shape of injectable mold. Other exemplary plastic materials may include, without limitation, BIOPAR FG MO (i.e., bio-plastic resin consisting mainly of thermoplastic potato starch, biodegradable synthetic copolyesters and additivies), BIOPLAST (i.e., new kind of plasticizer cherfreien thermoplastic material), ENSO RENEW RTP (i.e., renewable, biodegradable, compostable and economic thermoplastic), and/or the like. In one embodiment, device 144 is included of at least 50% biodegradable/compostable plastics as described herein by volume.

With continued reference to FIG. 5, outer body 502 may include PCB 504 containing NFC chip 506 connected with one or more antennas 508 as described above with reference to FIGS. 2A-B. One end of outer body 502 may be enclosed by a body base 510. As used in this disclosure, a “body base” is a chassis of aerosol delivery device 500. In some cases, body base 510 may include a body base seal 512, wherein the body base seal 512 is a component that seals the connection between outer body 502 and body base 510, preventing leaks and ensuring proper functioning of aerosol delivery device 500. In a non-limiting example, body base seal 512 may create a tight seal when pressed against bottom of aerosol delivery device 500.

With continued reference to FIG. 5, in other cases, body base 510 may include a base plug 514 connected to PCB 504, wherein the base plug 514 may include, without limitation, a transmitter, a separate PCB, a pressure sensor, a light element, and/or the like; for instance, base plug 514 may include a separate PCB with integrated pressure sensor. For another instance, and without limitation, base plug 514 may also include a base light (e.g., a status indicator continuously indicates one or more status of aerosol delivery device 500). In a non-limiting example, status indicator may include a liquid fill level indicator, internal condition indicator, charging indicator, and/or the like. Additionally, or alternatively, base plug 514 may include a lighting scheme, wherein the lighting scheme may include one or more openings that allow light to shine through. In some cases, lighting scheme may include an opening in a shape of a logo or a shape of an initial of company producing aerosol delivery device 500. Further, a mouthpiece 516 may fit into an opposite end of the end of aerosol delivery device 500 sealed by body base 510.

With continued reference to FIG. 5, in some cases, cartridge may be integrated into the device. In a non-limiting example, at least one reservoir 518 may be encased by outer body 502. Outer body 502, body base 510, and mouthpiece 516 may form an enclosure such that the at least one reservoir 518, along with power source 152 may be securely contained within the device. In some cases, power source 152 may include any battery as described above with reference to FIGS. 1A-D that contains one or more cell chemistries e.g., lithium cobalt oxide (LCO), lithium nickel cobalt aluminum oxide (NCA), lithium nickel manganese cobalt oxide (NMC), lithium iron phosphate (LFP), and the like.

In some cases, reservoir 518 may include a channel 522, wherein the channel 522 is a pathway or a passage through which aerosolized material flows. 522 is also encased by a cotton absorption pad 524 (i.e., reservoir cotton), centered around 522. Channel 522 may either be molded (i.e., injection molding) into the reservoir as an extension of a vapor tube 526 or may be separate components. Vapor tube 526 may either be molded as part of reservoir or be made of a different material and inserted later on. It's function is to transport aerosolized material from the heating chamber to the user. In a non-limiting example reservoir 518 may be in fluidic connection with heating element 530 such as, without limitation, a heating coil (i.e., a wire coil that heated to vaporize the aerosolizable material).

With continued reference to FIG. 5, a vapor channel seal 528 may be placed at the base of vapor tube 526 and encased the sides of heating element 530 to assist controlling of wicking and liquid flow into the heating chamber. A “vapor channel seal,” as described herein, is a sealing component in aerosol delivery device 500 that ensures an airtight seal and leak-proof seal within vapor path or airway. In an embodiment, a vapor channel seal 528 may be around the coil assembly (heating element 530). A heating coil cotton 532 may be wrapped around or threaded through the heating coil, ensuring that the aerosolizable material comes into contact with the heated coil when apparatus is activated. Heating coil cotton 532 may absorb aerosolizable material, and as the heating coil heats up, vaporizing the aerosolizable material, which may be then inhaled by the user. In a non-limiting example, heating coil cotton 532 may include a wick. In some cases, vapor channel seal 528 may also be configured to perform the function of wicking/funneling control similar to heating coil cotton 532. Additionally, or alternatively, heating element 530, vapor channel seal 528, and heating coil cotton 532 may be disposed inside reservoir 518 isolated from the aerosolizable material. Further, vapor channel seal 528 may serve as a seal with vapor tube; However, it also forms an aerosolization chamber when vapor channel seal 528 is inserted onto heating element 530 connected with the reservoir base 536 (i.e., liquid chamber deck).

With continued reference to FIG. 5, a reservoir base 536 may connect to reservoir 518. As used in this disclosure, a “reservoir base” is the base section of reservoir 518 which connected to heating element 530 (i.e., heating coil) and allows the wicking material such as, without limitation, heating coil cotton 532 to absorb aerosolizable material and deliver it to heating element 530 for vaporization. In a non-limiting example, reservoir base 536 with or without heating element 530, vapor channel seal 528, and/or heating coil cotton 532 attached may be inserted into reservoir 518 in a direction consistent with body base 510, along with a reservoir base seal 538, wherein the reservoir base seal 538 serves to prevent aerosolizable material from leaking out of reservoir 518 onto reservoir base 536 or other internal components such as, without limitation, power source 152, PCB 504, and/or the like. Additionally, or alternatively, a reservoir battery seal 540 may be disposed in between reservoir 518 and power source 152 (i.e., under reservoir base 528 and above power source 152), wherein the reservoir battery seal 540 serve as a secondary protection for power source 152, preventing aerosolizable material from leaking out through reservoir base 536 into power source 152.

With continued reference to FIG. 5, in some cases, instead of replacing cartridge, at least one reservoir 518 may be refilled. In an embodiment, at least one reservoir 518 may include a reservoir fill port seal 542 sealing a reservoir fill port 544, wherein the reservoir fill port 544 is a small opening on reservoir 518 and/or outer body 502 of aerosol delivery device 500 that allows user to fill reservoir 518 with user-preferred aerosolizable material. In some cases, reservoir fill port may be located on the top of reservoir 518 and covered by reservoir fill port seal 542. Reservoir fill port seal may be configured to prevent aerosolizable material from leaking out of the reservoir fill port and onto aerosol delivery device 500. In some cases, reservoir fill port seal 542 may include a removable cap or plug. Once reservoir 518 is filled, reservoir fill port seal 542 may be placed into reservoir fill port 544, sealing the reservoir fill port 544 from the inside.

With continued reference to FIG. 5, reservoir 518 may further include a reservoir seal 546 disposed at the opposite end of reservoir base seal 538. In a non-limiting example, reservoir seal 546 may be placed around reservoir fill port seal 542 and reservoir fill port 544. Snapping of mouthpiece 516 onto reservoir 518 may allow for both airflow management and avoiding condensation to seep out by configuring an airtight seal on top of reservoir 518. Airtight sealing both on top of reservoir 518 through reservoir seal 540 and bottom through reservoir base seal 538 may improve stability of active ingredient filled in reservoir 518 as it avoids contact with air (i.e., potential oxidation).

With continued reference to FIG. 5, cotton absorption pad 524 wrapped around the outlet of channel 522, may be referred to as a “reservoir cotton,” as described herein, is a component configured to absorb any excess aerosolizable material may have been vaporized by heating element 530 but not inhaled by the user, preventing any aerosolizable material from entering the user's mouth through mouthpiece 516. Further, cotton stand 548 may also be mechanically connected to mouthpiece 516 and hold a further cotton such as, without limitation, a mouthpiece cotton 552. Mouthpiece cotton 552 may be fixed on top of cotton stand inside mouthpiece 516. In an embodiment, mouthpiece cotton 552 may be in contact with the outlet of mouthpiece 516 and may be used as a filter configured to help prevent aerosolizable material from entering the user's mouth. In some cases, mouthpiece cotton 552 may also help to reduce condensation and improve the overall vaping experience.

With continued reference to FIG. 5, reservoir 518 may include a plurality of alignment features 554a-d on the exterior. As used in this disclosure, an “alignment feature” on the exterior of reservoir 518 is a physical feature that helps to precisely and securely align and/or fix reservoir 518 within outer body 502. In a non-limiting example, reservoir 518 may be internally coupled to outer body 502 through plurality of alignment features 554a-d. In some cases, alignment feature may include one or more male alignment features 554a-b, wherein the male alignment features 554a-b may include physical features that projects outwardly from reservoir 518, while the female alignment features 554c-d may include corresponding physical feature that is recessed or indented into reservoir 518, designed to receive and align with male alignment features 554a-b. In a non-limiting example, reservoir 518 may be inserted into outer body 502 through press fit and/or snap fit. The interior of outer body may include a plurality of alignment features that match plurality of alignment features 554a-d on the reservoir 518. For instance, and without limitation, female alignment features 554c-d may include windows around reservoir 518, wherein these windows may be configured to fit plurality of male alignment features (e.g., bumps or protrusions) within outer body 502 at a desired location.

With continued reference to FIG. 5, aerosol delivery device 500 may include a top/bottom seal 556a-b, wherein the top/bottom seal 556a-b. Top seal 556a may be placed over (e.g., covering) the mouthpiece 516 while bottom seal 556b may be placed over end cap 510 and some portion of outer body 502 towards end cap 510. In some cases, during fluid e.g., air or vaporized aerosolizable material travel tight top/bottom seal 556a-b, such seal may help to stabilize the pressure changes and prevent any leakage that may occur. In an embodiment, one or more rubber extrusions/inserts (within top/bottom seal 556a-b) may help further create an airtight seal by inserting the extrusions/inserts into connecting components (e.g., mouthpiece 516, end cap 510, and/or the like).

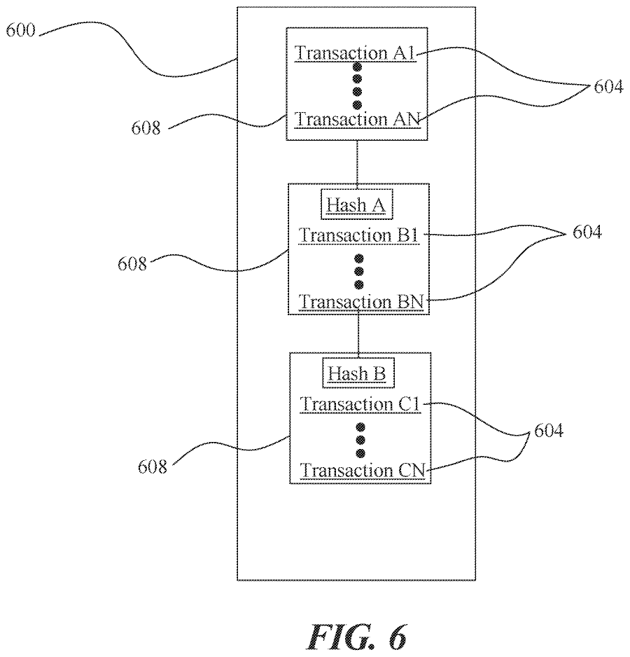

Referring now to FIG. 6, an exemplary embodiment of an immutable sequential listing is illustrated. Data elements listed in immutable sequential listing may include any form of data, including textual data, image data, encrypted data, cryptographically hashed data, and the like. Data elements may include, without limitation, one or more at least a digitally signed assertion. In one embodiment, a digitally signed assertion 604 is a collection of textual data signed using a secure proof as described in further detail below; secure proof may include, without limitation, a digital signature as described above. Collection of textual data may contain any textual data, including without limitation American Standard Code for Information Interchange (ASCII), Unicode, or similar computer-encoded textual data, any alphanumeric data, punctuation, diacritical mark, or any character or other marking used in any writing system to convey information, in any form, including any plaintext or cyphertext data; in an embodiment, collection of textual data may be encrypted, or may be a hash of other data, such as a root or node of a Merkle tree or hash tree, or a hash of any other information desired to be recorded in some fashion using a digitally signed assertion 604. In an embodiment, collection of textual data states that the owner of a certain transferable item represented in a digitally signed assertion 604 register is transferring that item to the owner of an address. A digitally signed assertion 604 may be signed by a digital signature created using the private key associated with the owner's public key, as described above.

With continued reference to FIG. 6, a digitally signed assertion 604 may describe a transfer of virtual currency, such as crypto-currency as described below. The virtual currency may be a digital currency. Item of value may be a transfer of trust, for instance represented by a statement vouching for the identity or trustworthiness of the first entity. Item of value may be an interest in a fungible negotiable financial instrument representing ownership in a public or private corporation, a creditor relationship with a governmental body or a corporation, rights to ownership represented by an option, derivative financial instrument, commodity, debt-backed security such as a bond or debenture or other security as described in further detail below. A resource may be a physical machine e.g., a ride share vehicle or any other asset. A digitally signed assertion 604 may describe the transfer of a physical good; for instance, a digitally signed assertion 604 may describe the sale of a product. In some embodiments, a transfer nominally of one item may be used to represent a transfer of another item; for instance, a transfer of virtual currency may be interpreted as representing a transfer of an access right; conversely, where the item nominally transferred is something other than virtual currency, the transfer itself may still be treated as a transfer of virtual currency, having value that depends on many potential factors including the value of the item nominally transferred and the monetary value attendant to having the output of the transfer moved into a particular user's control. The item of value may be associated with a digitally signed assertion 604 by means of an exterior protocol, such as the COLORED COINS created according to protocols developed by The Colored Coins Foundation, the MASTERCOIN protocol developed by the Mastercoin Foundation, or the ETHEREUM platform offered by the Stiftung Ethereum Foundation of Baar, Switzerland, the Thunder protocol developed by Thunder Consensus, or any other protocol.

Still referring to FIG. 6, in one embodiment, an address is a textual datum identifying the recipient of virtual currency or another item of value in a digitally signed assertion 604. In some embodiments, address is linked to a public key, the corresponding private key of which is owned by the recipient of a digitally signed assertion 604. For instance, address may be the public key. Address may be a representation, such as a hash, of the public key. Address may be linked to the public key in memory of a computing device, for instance via a “wallet shortener” protocol. Where address is linked to a public key, a transferee in a digitally signed assertion 604 may record a subsequent a digitally signed assertion 604 transferring some or all of the value transferred in the first a digitally signed assertion 604 to a new address in the same manner. A digitally signed assertion 604 may contain textual information that is not a transfer of some item of value in addition to, or as an alternative to, such a transfer. For instance, as described in further detail below, a digitally signed assertion 604 may indicate a confidence level associated with a distributed storage node as described in further detail below.

With continued reference to FIG. 6, in an embodiment, immutable sequential listing records a series of at least a posted content in a way that preserves the order in which the at least a posted content took place. Temporally sequential listing may be accessible at any of various security settings; for instance, and without limitation, temporally sequential listing may be readable and modifiable publicly, may be publicly readable but writable only by entities and/or devices having access privileges established by password protection, confidence level, or any device authentication procedure or facilities described herein, or may be readable and/or writable only by entities and/or devices having such access privileges. Access privileges may exist in more than one level, including, without limitation, a first access level or community of permitted entities and/or devices having ability to read, and a second access level or community of permitted entities and/or devices having ability to write; first and second community may be overlapping or non-overlapping. In an embodiment, posted content and/or immutable sequential listing may be stored as one or more zero knowledge sets (ZKS), Private Information Retrieval (PIR) structure, or any other structure that allows checking of membership in a set by querying with specific properties. Such database may incorporate protective measures to ensure that malicious actors may not query the database repeatedly in an effort to narrow the members of a set to reveal uniquely identifying information of a given posted content.

With continued reference to FIG. 6, immutable sequential listing may preserve the order in which the at least a posted content took place by listing them in chronological order; alternatively or additionally, immutable sequential listing may organize digitally signed assertions 604 into sub-listings 608 such as “blocks” in a blockchain, which may be themselves collected in a temporally sequential order; digitally signed assertions 604 within a sub-listing 608 may or may not be temporally sequential. The ledger may preserve the order in which at least a posted content took place by listing them in sub-listings 608 and placing the sub-listings 608 in chronological order. The immutable sequential listing may be a distributed, consensus-based ledger, such as those operated according to the protocols promulgated by Ripple Labs, Inc., of San Francisco, Calif., or the Stellar Development Foundation, of San Francisco, Calif, or of Thunder Consensus. In some embodiments, the ledger is a secured ledger; in one embodiment, a secured ledger is a ledger having safeguards against alteration by unauthorized parties. The ledger may be maintained by a proprietor, such as a system administrator on a server, that controls access to the ledger; for instance, the user account controls may allow contributors to the ledger to add at least a posted content to the ledger but may not allow any users to alter at least a posted content that have been added to the ledger. In some embodiments, ledger is cryptographically secured; in one embodiment, a ledger is cryptographically secured where each link in the chain contains encrypted or hashed information that makes it practically infeasible to alter the ledger without betraying that alteration has taken place, for instance by requiring that an administrator or other party sign new additions to the chain with a digital signature. Immutable sequential listing may be incorporated in, stored in, or incorporate, any suitable data structure, including without limitation any database, datastore, file structure, distributed hash table, directed acyclic graph or the like. In some embodiments, the timestamp of an entry is cryptographically secured and validated via trusted time, either directly on the chain or indirectly by utilizing a separate chain. In one embodiment the validity of timestamp is provided using a time stamping authority as described in the RFC 6161 standard for trusted timestamps, or in the ANSI ASC x9.95 standard. In another embodiment, the trusted time ordering is provided by a group of entities collectively acting as the time stamping authority with a requirement that a threshold number of the group of authorities sign the timestamp.

In some embodiments, and with continued reference to FIG. 6, immutable sequential listing, once formed, may be inalterable by any party, no matter what access rights that party possesses. For instance, immutable sequential listing may include a hash chain, in which data is added during a successive hashing process to ensure non-repudiation. Immutable sequential listing may include a block chain. In one embodiment, a block chain is immutable sequential listing that records one or more new at least a posted content in a data item known as a sub-listing 608 or “block.” An example of a block chain is the BITCOIN block chain used to record BITCOIN transactions and values. Sub-listings 608 may be created in a way that places the sub-listings 608 in chronological order and link each sub-listing 608 to a previous sub-listing 608 in the chronological order so that any computing device may traverse the sub-listings 608 in reverse chronological order to verify any at least a posted content listed in the block chain. Each new sub-listing 608 may be required to contain a cryptographic hash describing the previous sub-listing 608. In some embodiments, the block chain contains a single first sub-listing 608 sometimes known as a “genesis block.”

Still referring to FIG. 6, the creation of a new sub-listing 608 may be computationally expensive; for instance, the creation of a new sub-listing 608 may be designed by a “proof of work” protocol accepted by all participants in forming the immutable sequential listing to take a powerful set of computing devices a certain period of time to produce. Where one sub-listing 608 takes less time for a given set of computing devices to produce the sub-listing 608 protocol may adjust the algorithm to produce the next sub-listing 608 so that it will require more steps; where one sub-listing 608 takes more time for a given set of computing devices to produce the sub-listing 608 protocol may adjust the algorithm to produce the next sub-listing 608 so that it will require fewer steps. As an example, protocol may require a new sub-listing 608 to contain a cryptographic hash describing its contents; the cryptographic hash may be required to satisfy a mathematical condition, achieved by having the sub-listing 608 contain a number, called a nonce, whose value is determined after the fact by the discovery of the hash that satisfies the mathematical condition. Continuing the example, the protocol may be able to adjust the mathematical condition so that the discovery of the hash describing a sub-listing 608 and satisfying the mathematical condition requires more or less steps, depending on the outcome of the previous hashing attempt. Mathematical condition, as an example, might be that the hash contains a certain number of leading zeros and a hashing algorithm that requires more steps to find a hash containing a greater number of leading zeros, and fewer steps to find a hash containing a lesser number of leading zeros. In some embodiments, production of a new sub-listing 608 according to the protocol is known as “mining.” The creation of a new sub-listing 608 may be designed by a “proof of stake” protocol as will be apparent to those skilled in the art upon reviewing the entirety of this disclosure.

Continuing to refer to FIG. 6, in some embodiments, protocol also creates an incentive to mine new sub-listings 608. The incentive may be financial; for instance, successfully mining a new sub-listing 608 may result in the person or entity that mines the sub-listing 608 receiving a predetermined amount of currency. The currency may be fiat currency. Currency may be cryptocurrency as defined below. In other embodiments, incentive may be redeemed for particular products or services; the incentive may be a gift certificate with a particular business, for instance. In some embodiments, incentive is sufficiently attractive to cause participants to compete for the incentive by trying to race each other to the creation of sub-listings 608 Each sub-listing 608 created in immutable sequential listing may contain a record or at least a posted content describing one or more addresses that receive an incentive, such as virtual currency, as the result of successfully mining the sub-listing 608.

With continued reference to FIG. 6, where two entities simultaneously create new sub-listings 608, immutable sequential listing may develop a fork; protocol may determine which of the two alternate branches in the fork is the valid new portion of the immutable sequential listing by evaluating, after a certain amount of time has passed, which branch is longer. “Length” may be measured according to the number of sub-listings 608 in the branch. Length may be measured according to the total computational cost of producing the branch. Protocol may treat only at least a posted content contained the valid branch as valid at least a posted content. When a branch is found invalid according to this protocol, at least a posted content registered in that branch may be recreated in a new sub-listing 608 in the valid branch; the protocol may reject “double spending” at least a posted content that transfer the same virtual currency that another at least a posted content in the valid branch has already transferred. As a result, in some embodiments the creation of fraudulent at least a posted content requires the creation of a longer immutable sequential listing branch by the entity attempting the fraudulent at least a posted content than the branch being produced by the rest of the participants; as long as the entity creating the fraudulent at least a posted content is likely the only one with the incentive to create the branch containing the fraudulent at least a posted content, the computational cost of the creation of that branch may be practically infeasible, guaranteeing the validity of all at least a posted content in the immutable sequential listing.

Still referring to FIG. 6, additional data linked to at least a posted content may be incorporated in sub-listings 608 in the immutable sequential listing; for instance, data may be incorporated in one or more fields recognized by block chain protocols that permit a person or computer forming a at least a posted content to insert additional data in the immutable sequential listing. In some embodiments, additional data is incorporated in an unspendable at least a posted content field. For instance, the data may be incorporated in an OP RETURN within the BITCOIN block chain. In other embodiments, additional data is incorporated in one signature of a multi-signature at least a posted content. In an embodiment, a multi-signature at least a posted content is at least a posted content to two or more addresses. In some embodiments, the two or more addresses are hashed together to form a single address, which is signed in the digital signature of the at least a posted content. In other embodiments, the two or more addresses are concatenated. In some embodiments, two or more addresses may be combined by a more complicated process, such as the creation of a Merkle tree or the like. In some embodiments, one or more addresses incorporated in the multi-signature at least a posted content are typical crypto-currency addresses, such as addresses linked to public keys as described above, while one or more additional addresses in the multi-signature at least a posted content contain additional data related to the at least a posted content; for instance, the additional data may indicate the purpose of the at least a posted content, aside from an exchange of virtual currency, such as the item for which the virtual currency was exchanged. In some embodiments, additional information may include network statistics for a given node of network, such as a distributed storage node, e.g. the latencies to nearest neighbors in a network graph, the identities or identifying information of neighboring nodes in the network graph, the trust level and/or mechanisms of trust (e.g. certificates of physical encryption keys, certificates of software encryption keys, (in non-limiting example certificates of software encryption may indicate the firmware version, manufacturer, hardware version and the like), certificates from a trusted third party, certificates from a decentralized anonymous authentication procedure, and other information quantifying the trusted status of the distributed storage node) of neighboring nodes in the network graph, IP addresses, GPS coordinates, and other information informing location of the node and/or neighboring nodes, geographically and/or within the network graph. In some embodiments, additional information may include history and/or statistics of neighboring nodes with which the node has interacted. In some embodiments, this additional information may be encoded directly, via a hash, hash tree or other encoding.

With continued reference to FIG. 6, in some embodiments, virtual currency is traded as a crypto-currency. In one embodiment, a crypto-currency is a digital, currency such as Bitcoins, Peercoins, Namecoins, and Litecoins. Crypto-currency may be a clone of another crypto-currency. The crypto-currency may be an “alt-coin.” Crypto-currency may be decentralized, with no particular entity controlling it; the integrity of the crypto-currency may be maintained by adherence by its participants to established protocols for exchange and for production of new currency, which may be enforced by software implementing the crypto-currency. Crypto-currency may be centralized, with its protocols enforced or hosted by a particular entity. For instance, crypto-currency may be maintained in a centralized ledger, as in the case of the) M) currency of Ripple Labs, Inc., of San Francisco, Calif. In lieu of a centrally controlling authority, such as a national bank, to manage currency values, the number of units of a particular crypto-currency may be limited; the rate at which units of crypto-currency enter the market may be managed by a mutually agreed-upon process, such as creating new units of currency when mathematical puzzles are solved, the degree of difficulty of the puzzles being adjustable to control the rate at which new units enter the market. Mathematical puzzles may be the same as the algorithms used to make productions of sub-listings 608 in a block chain computationally challenging; the incentive for producing sub-listings 608 may include the grant of new crypto-currency to the miners. Quantities of crypto-currency may be exchanged using at least a posted content as described above.

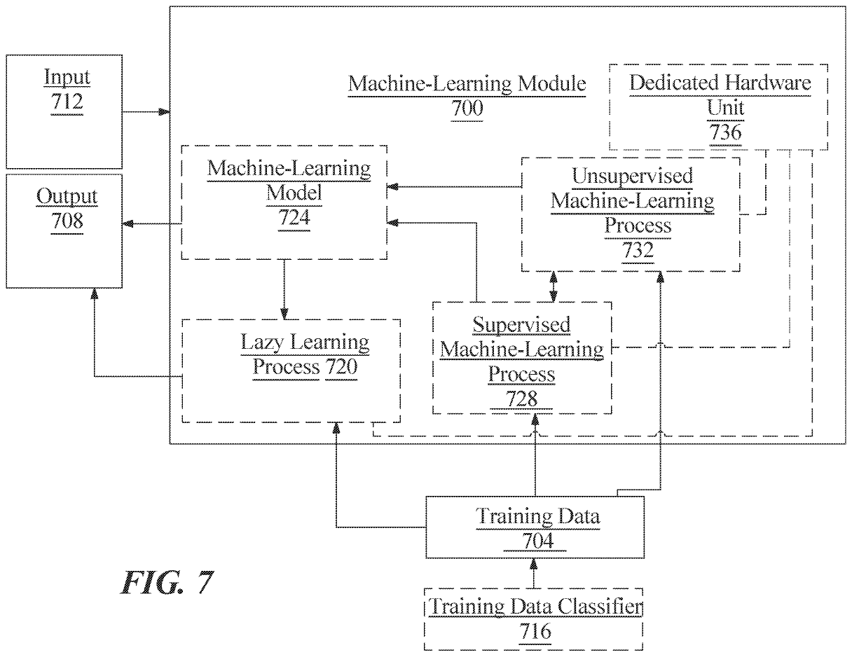

Referring now to FIG. 7, an exemplary embodiment of a machine-learning module 700 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 704 to generate an algorithm instantiated in hardware or software logic, data structures, and/or functions that will be performed by a computing device/module to produce outputs 708 given data provided as inputs 712; this is in contrast to a non-machine learning software program where the commands to be executed are determined in advance by a user and written in a programming language.

Still referring to FIG. 7, “training data,” as used herein, is data containing correlations that a machine-learning process may use to model relationships between two or more categories of data elements. For instance, and without limitation, training data 704 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 704 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 704 according to various correlations; correlations may indicate causative and/or predictive links between categories of data elements, which may be modeled as relationships such as mathematical relationships by machine-learning processes as described in further detail below. Training data 704 may be formatted and/or organized by categories of data elements, for instance by associating data elements with one or more descriptors corresponding to categories of data elements. As a non-limiting example, training data 704 may include data entered in standardized forms by persons or processes, such that entry of a given data element in a given field in a form may be mapped to one or more descriptors of categories. Elements in training data 704 may be linked to descriptors of categories by tags, tokens, or other data elements; for instance, and without limitation, training data 704 may be provided in fixed-length formats, formats linking positions of data to categories such as comma-separated value (CSV) formats and/or self-describing formats such as extensible markup language (XML), Javascript Object Notation (JSON), or the like, enabling processes or devices to detect categories of data.

Alternatively, or additionally, and continuing to refer to FIG. 7, training data 704 may include one or more elements that are not categorized; that is, training data 704 may not be formatted or contain descriptors for some elements of data. Machine-learning algorithms and/or other processes may sort training data 704 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 704 to be made applicable for two or more distinct machine-learning algorithms as described in further detail below. Training data 704 used by machine-learning module 700 may correlate any input data as described in this disclosure to any output data as described in this disclosure. As a non-limiting illustrative example training data may include a plurality of sensed datums and/or unique identifiers as input, correlated to a plurality of authentication datums as output.

Further referring to FIG. 7, training data may be filtered, sorted, and/or selected using one or more supervised and/or unsupervised machine-learning processes and/or models as described in further detail below; such models may include without limitation a training data classifier 716. Training data classifier 716 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 700 may generate a classifier using a classification algorithm, defined as a processes whereby a computing device and/or any module and/or component operating thereon derives a classifier from training data 704. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 7, computing device 704 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)±1³ (B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 704 may then calculate a likelihood table by calculating probabilities of different data entries and classification labels. Computing device 704 may utilize a naive Bayes equation to calculate a posterior probability for each class. A class containing the highest posterior probability is the outcome of prediction. Naive Bayes classification algorithm may include a gaussian model that follows a normal distribution. Naive Bayes classification algorithm may include a multinomial model that is used for discrete counts. Naïve Bayes classification algorithm may include a Bernoulli model that may be utilized when vectors are binary.

With continued reference to FIG. 7, computing device 704 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 7, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: I=aᵢ², where α, is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 7, training examples for use as training data may be selected from a population of potential examples according to cohorts relevant to an analytical problem to be solved, a classification task, or the like. Alternatively, or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively, or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 7, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 7, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively, or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 7, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 7, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 7, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 7, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 7, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ, :

$${x}_{n e w} = \frac{X - {X}_{mix}}{{X}_{m ax} - {X}_{m i n}} .$$

Feature scaling may include mean normalization, which involves use of a mean A max Xmtn value of a set and/or subset of values, Xₘₑₐₙ with maximum and minimum values:

$${X}_{n e w} = \frac{X - {X}_{m e a n}}{{X}_{m ax} - {X}_{m i n}} .$$

Feature scaling may include standardization, where a difference between X and Xₘₑₐₙ is divided by a is standard deviation a of σ set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25^{th} percentile value and the 5e percentile value (or closest values thereto by a rounding protocol), such as:

$${X}_{n e w} = \frac{X - {X}_{median}}{IQR}$$

Persons skilled in the art, upon reviewing the entirety of this disclosure, will be aware of various alternative or additional approaches that may be used for feature scaling.

Still referring to FIG. 7, machine-learning module 700 may be configured to perform a lazy-learning process 720 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 704. Heuristic may include selecting some number of highest-ranking associations and/or training data 704 elements. Lazy learning may implement any suitable lazy learning algorithm, including without limitation a K-nearest neighbors algorithm, a lazy naive Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 7, machine-learning processes as described in this disclosure may be used to generate machine-learning models 724. A “machine-learning model,” as used in this disclosure, is a data structure representing and/or instantiating a mathematical and/or algorithmic representation of a relationship between inputs and outputs, as generated using any machine-learning process including without limitation any process as described above and stored in memory; an input is submitted to a machine-learning model 724 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 724 may be generated by creating an artificial neural network, such as a convolutional neural network comprising an input layer of nodes, one or more intermediate layers, and an output layer of nodes. Connections between nodes may be created via the process of “training” the network, in which elements from a training data 704 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 7, machine-learning algorithms may include at least a supervised machine-learning process 728. At least a supervised machine-learning process 728, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include plurality of sensed datums and/or unique identifiers as described above as inputs, plurality of authentication datums as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 704. Persons skilled in the art, upon reviewing the entirety of this disclosure, will be aware of various possible variations of at least a supervised machine-learning process 728 that may be used to determine relation between inputs and outputs. Supervised machine-learning processes may include classification algorithms as defined above.

With further reference to FIG. 7, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively, or additionally, one or more errors and/or error function values evaluated in training iterations may be compared to a threshold.

Still referring to FIG. 7, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 7, machine learning processes may include at least an unsupervised machine-learning processes 732. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 732 may not require a response variable; unsupervised processes 732 may be used to find interesting patterns and/or inferences between variables, to determine a degree of correlation between two or more variables, or the like.

Still referring to FIG. 7, machine-learning module 700 may be designed and configured to create a machine-learning model 724 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 7, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 7, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 7, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 7, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 7, one or more processes or algorithms described above may be performed by at least a dedicated hardware unit 736. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 736 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 736 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 736 to perform one or more operations described herein, such as evaluation of model and/or algorithm outputs, one-time or iterative updates to parameters, coefficients, weights, and/or biases, and/or any other operations such as vector and/or matrix operations as described in this disclosure.

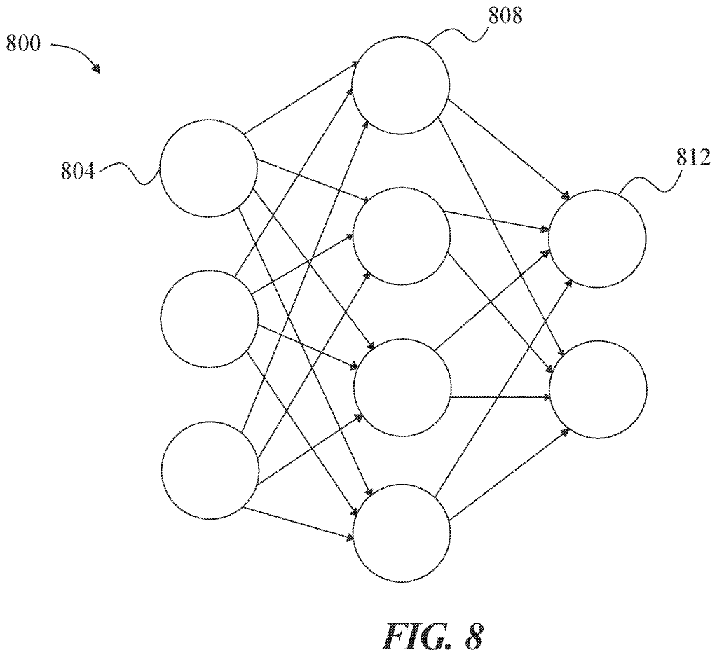

Referring now to FIG. 8, an exemplary embodiment of neural network 800 is illustrated. A neural network 800 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 804, one or more intermediate layers 808, and an output layer of nodes 812. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

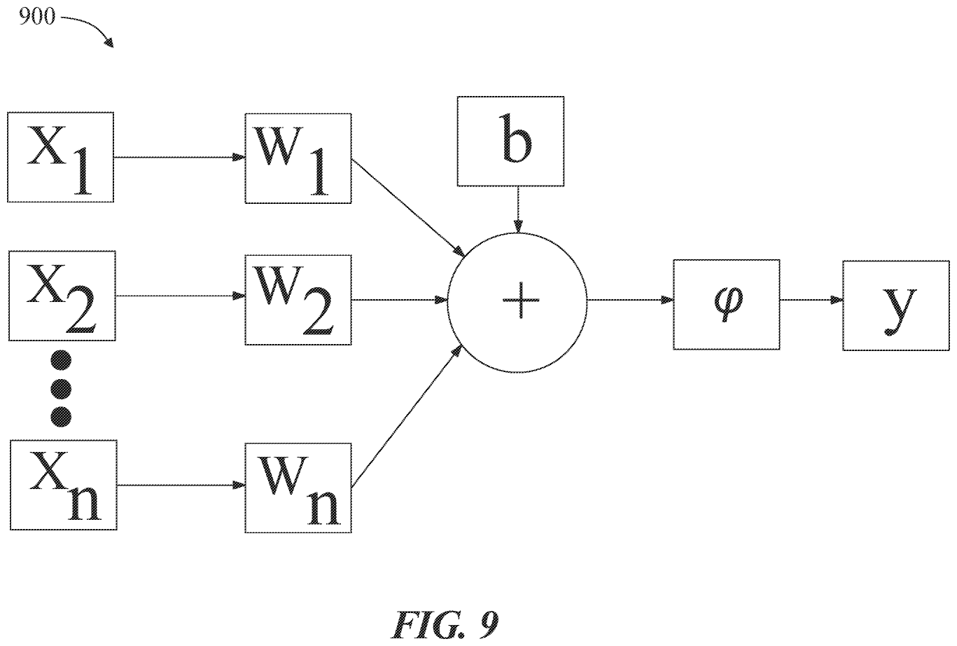

Referring now to FIG. 9, an exemplary embodiment of a node 900 of a neural network is illustrated. A node may include, without limitation a plurality of inputs x, that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh² (x), a rectified linear unit function such as ƒ(x)=max (0, x), a “leaky” and/or “parametric” rectified linear unit function such as ƒ(x)=max (ax, x) for some α, an exponential linear units function such as

$$f ( x ) = \{ \begin{matrix}x for x \geq 0 \\ a ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid (x), a Gaussian error linear unit function such as ƒ(x)=a (1+tanh (\sqrt{2/π}(x+bx^{r}))) for some values of a, b, and r, and/or a scaled exponential linear unit function such as

$$f ( x ) = λ \{ \begin{matrix}a ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function co, which may generate one or more outputs y. Weight wᵢ applied to an input z may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wi may be determined by training a neural network using training data, which may be performed using any suitable process as described above.

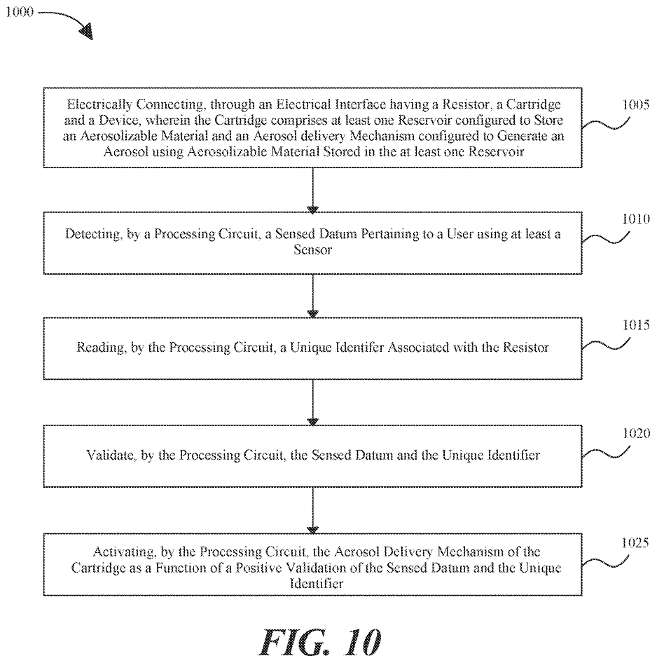

Now referring to FIG. 10, a flow diagram of an exemplary embodiment of a method 1000 for preventing counterfeit aerosol delivery is illustrated. The method 1000 includes a step 1005 of electrically connecting, through an electrical interface having a resistor, a cartridge and a device, wherein the cartridge includes at least one reservoir configured to store an aerosolizable material and an aerosol delivery mechanism configured to generate aerosol using the aerosolizable material stored in the at least one reservoir, and wherein the device comprises at least a sensor. In some embodiments, the resistor may include a surface mount device (SMD) resistor. In some embodiments, the at least a sensor may include a biometric sensor. This may be implemented, without limitation, as described above with reference to FIGS. 1-9.

With continued reference to FIG. 10, the method 1000 includes a step 1010 of detecting, by a processing circuit, a sensed datum pertaining to a user using the at least a sensor. In some embodiments, the sensed datum may include at least a biometric identifier of the user detected by the biometric sensor. In other embodiments, the sensed datum may include user behavior data. This may be implemented, without limitation, as described above with reference to FIGS. 1-9.

With continued reference to FIG. 10, the method 1000 includes a step 1015 of reading, by the processing circuit, a unique identifier associated with the resistor. In some embodiments, the unique identifier may include a resistance value. This may be implemented, without limitation, as described above with reference to FIGS. 1-9.

With continued reference to FIG. 10, the method 1000 includes a step 1020 of validating, by the processing circuit, the sensed datum, and the unique identifier. In some embodiments, validating the sensed datum and the unique identifier may include recognizing the cartridge as a function of the unique identifier associated with the resistor by cross-referencing the unique identifier with a database of approved unique identifiers. In some embodiments, validating the sensed datum and the unique identifier further include comparing the sensed datum against a user profile pertaining to the user, and generating an authentication datum upon a positive match of both the unique identifier and the sensed datum. This may be implemented, without limitation, as described above with reference to FIGS. 1-9.

With continued reference to FIG. 10, the method 1000 includes a step 1025 of activating, by the processing circuit, the aerosol delivery mechanism of the cartridge as a function of a positive validation of the sensed datum and the unique identifier. In some embodiments, activating the aerosol delivery mechanism of the cartridge may include activating the aerosol delivery mechanism as a function of the authentication datum. This may be implemented, without limitation, as described above with reference to FIGS. 1-9.

With continued reference to FIG. 10, the method 1000 may further include a step of communicating, using a wireless communication device, the sensed datum and the unique identifier to an external device, wherein the wireless communication device includes a near field communication (NFC) chip, and an antenna communicatively connected to the NFC chip.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

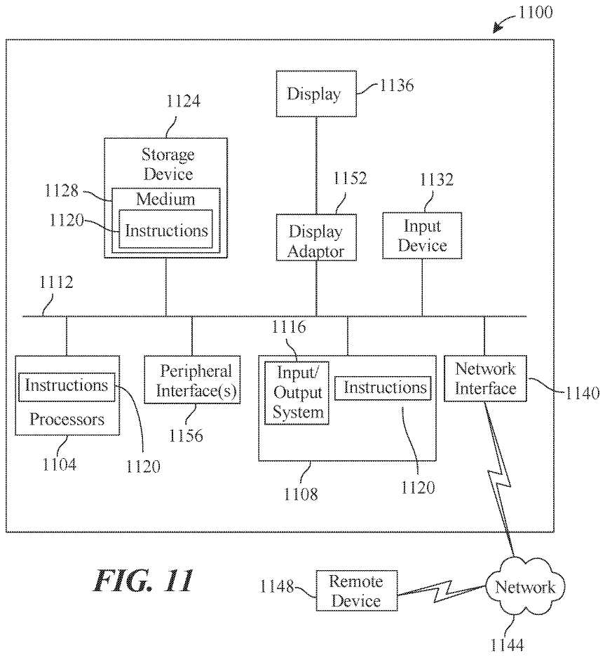

FIG. 11 shows a diagrammatic representation of one embodiment of a computing device in the exemplary form of a computer system 1100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1100 includes a processor 1104 and a memory 1108 that communicate with each other, and with other components, via a bus 1112. Bus 1112 may include any of several types of bus structures including, but not limited to, a memory bus, a memory controller, a peripheral bus, a local bus, and any combinations thereof, using any of a variety of bus architectures.

Processor 1104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1104 may be organized according to Von Neumann and/or Harvard architecture as a non-limiting example. Processor 1104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1108 may include various components (e.g., machine-readable media) including, but not limited to, a random-access memory component, a read only component, and any combinations thereof. In one example, a basic input/output system 1116 (BIOS), including basic routines that help to transfer information between elements within computer system 1100, such as during start-up, may be stored in memory 1108. Memory 1108 may also include (e.g., stored on one or more machine-readable media) instructions (e.g., software) 1120 embodying any one or more of the aspects and/or methodologies of the present disclosure. In another example, memory 1108 may further include any number of program modules including, but not limited to, an operating system, one or more application programs, other program modules, program data, and any combinations thereof.

Computer system 1100 may also include a storage device 1124. Examples of a storage device (e.g., storage device 1124) include, but are not limited to, a hard disk drive, a magnetic disk drive, an optical disc drive in combination with an optical medium, a solid-state memory device, and any combinations thereof. Storage device 1124 may be connected to bus 1112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1124 (or one or more components thereof) may be removably interfaced with computer system 1100 (e.g., via an external port connector (not shown)). Particularly, storage device 1124 and an associated machine-readable medium 1128 may provide nonvolatile and/or volatile storage of machine-readable instructions, data structures, program modules, and/or other data for computer system 1100. In one example, software 1120 may reside, completely or partially, within machine-readable medium 1128. In another example, software 1120 may reside, completely or partially, within processor 1104.

Computer system 1100 may also include an input device 1132. In one example, a user of computer system 1100 may enter commands and/or other information into computer system 1100 via input device 1132. Examples of an input device 1132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1132 may be interfaced to bus 1112 via any of a variety of interfaces (not shown) including, but not limited to, a serial interface, a parallel interface, a game port, a USB interface, a FIREWIRE interface, a direct interface to bus 1112, and any combinations thereof. Input device 1132 may include a touch screen interface that may be a part of or separate from display 1136, discussed further below. Input device 1132 may be utilized as a user selection device for selecting one or more graphical representations in a graphical interface as described above.

A user may also input commands and/or other information to computer system 1100 via storage device 1124 (e.g., a removable disk drive, a flash drive, etc.) and/or network interface device 1140. A network interface device, such as network interface device 1140, may be utilized for connecting computer system 1100 to one or more of a variety of networks, such as network 1144, and one or more remote devices 1148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1144, may employ a wired and/or a wireless mode of communication. In general, any network topology may be used. Information (e.g., data, software 1120, etc.) may be communicated to and/or from computer system 1100 via network interface device 1140.

Computer system 1100 may further include a video display adapter 1152 for communicating a displayable image to a display device, such as display device 1136. Examples of a display device include, but are not limited to, a liquid crystal display (LCD), a cathode ray tube (CRT), a plasma display, a light emitting diode (LED) display, and any combinations thereof. Display adapter 1152 and display device 1136 may be utilized in combination with processor 1104 to provide graphical representations of aspects of the present disclosure. In addition to a display device, computer system 1100 may include one or more other peripheral output devices including, but not limited to, an audio speaker, a printer, and any combinations thereof. Such peripheral output devices may be connected to bus 1112 via a peripheral interface 1156. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for aerosol access control, wherein the system comprises:
an external device;
a cartridge comprising:
a reservoir configured to store an aerosolizable material,
an aerosol delivery mechanism configured to generate an aerosol using the aerosolizable material stored in the reservoir, and a unique identifier; and an aerosol delivery device comprising:
a biometric sensor,
an NFC or RFID chip, and a processing circuit communicatively connected to the biometric sensor and to the NFC or RFID chip, wherein the processing circuit is configured to: receive, via the NFC chip or RFID chip, an external response generated by an external device, initially activate the biometric sensor based on the external response, subsequent to the initial activation, detect a datum pertaining to a user using the biometric sensor, validate the datum pertaining to the user, read the unique identifier, validate the unique identifier, and activate the aerosol delivery mechanism as a function of a positive validation of the datum pertaining to the user and the unique identifier.

2. The system of claim 1, wherein the unique identifier is configured to distinguish one cartridge from another cartridge.

3. The system of claim 2, wherein the unique identifier comprises a value or set of values.

4. The system of claim 1, wherein the biometric sensor comprises a fingerprint scanner.

5. The system of claim 1, wherein the validation of the unique identifier comprises cross-referencing the unique identifier with a database of approved unique identifiers.

6. The system of claim 2, wherein the unique identifier comprises information identifying a cartridge characteristic including at least one of a flavor, a nicotine content, or a brand.

7. The system of claim 5, wherein the processing circuit is further configured to indicate a counterfeit or unauthorized product if the unique identifier does not match any entry in database.

8. The system of claim 1, wherein the external device is a point of sale device.

9. A method for aerosol access control, wherein the method comprising: providing an external device; providing a cartridge comprising:
a reservoir configured to store an aerosolizable material,
an aerosol delivery mechanism configured to generate an aerosol using the aerosolizable material stored in the reservoir, and a unique identifier; and an aerosol delivery device comprising:
a biometric sensor,
an NFC or RFID chip, and a processing circuit communicatively connected to the biometric sensor and to the NFC or RFID chip; receiving, via the NFC chip or RFID chip, an external response generated by an external device, initially activating the biometric sensor based on the external response, subsequent to the initial activation, detecting a datum pertaining to a user using the biometric sensor, validating the datum pertaining to the user, reading the unique identifier using the processing circuit, and activating the aerosol delivery mechanism as a function of a positive validation of the datum pertaining to the user and the unique identifier.

10. The method of claim 11, wherein the unique identifier is configured to distinguish one cartridge from another cartridge.

11. The method of claim 10, wherein the unique identifier comprises a value or set of values.

12. The method of claim 11, wherein the biometric sensor comprises a fingerprint scanner.

13. The method of claim 11, wherein the validation of the unique identifier comprises cross-referencing the unique identifier with a database of approved unique identifiers.

14. The method of claim 10, wherein the unique identifier comprises information identifying a cartridge characteristic including at least one of a flavor, a nicotine content, or a brand.

15. The method of claim 13, wherein the processing circuit is further configured to indicate a counterfeit or unauthorized product if the unique identifier does not match any entry in database.

16. The method of claim 11, wherein the external device is a point of sale device.

17. The system of claim 1, further comprising an electrical interface between the cartridge and the aerosol delivery device, configured to allow for communication therebetween.

18. The system of claim 17, the electrical interface comprising a unique pin layout.

19. The system of claim 18, the unique identifier comprising the unique pin layout.

20. The method of claim 11, wherein the reading of the unique identifier includes sensing a unique electrical pin layout of the cartridge.
